(19) United States
(12) Patent Application Publication
(10) Pub. No.: US 2023/0034359 A1
(43) Pub. Date: Feb. 2, 2023

(54) GENERATION OF MATURE KUPFFER CELLS

(71) Applicant: AGENCY FOR SCIENCE, TECHNOLOGY AND RESEARCH, Singapore (SG)

(72) Inventors: Farah TASNIM, Singapore (SG); Hanry YU, Singapore (SG)

(73) Assignee: AGENCY FOR SCIENCE, TECHNOLOGY AND RESEARCH, Singapore (SG)

(21) Appl. No.: 17/295,734

(22) PCT Filed: Nov. 16, 2019
(86) PCT No.: PCT/SG2019/050563

§ 371 Date: May 20, 2021

(30) Foreign Application Priority Data
Nov. 21, 2018 (SG) 10201810417U

(51) Int. Cl. C12N 5/0786; G01N 33/50

(52) U.S. Cl. CPC C12N 5/0645; G01N 33/5014; C12N 2501/125; C12N 2501/155; C12N 2501/165; C12N 2501/22; C12N 2501/2303; C12N 2502/1157; C12N 2502/14; C12N 2503/04

(57) ABSTRACT

The invention relates to a method of producing an iPSC-derived Kupffer Cell (IKC). The method may comprise providing a macrophage precursor (preMcp) derived from an induced pluripotent stem cell (iPSC). The macrophage precursor (preM-cp) may be cultured in the presence of a hepatic cue, such as a combination of primary human hepatocyte conditioned media and Advanced DMEM, thereby obtaining the iPSC-derived Kupffer Cell. The iPSC-derived Kupffer Cell may display a biological property of a primary Kupffer cell, such as a primary adult human KC (pKC). The biological activity comprises expression of a macrophage marker such as CD11, CD14, CD68, CD163, CD32, CLEC-4F, ID1 and ID3.

### FIELD

This invention relates to the fields of medicine, cell biology, molecular biology and genetics. This invention relates to the field of medicine.

### BACKGROUND

Kupffer cells (KCs) are innate immune cells in liver; and are specialized in performing scavenger and phagocytic functions [1]. They play a critical role in normal liver physiological homeostasis and contribute to the pathogenesis of different liver diseases such as liver fibrosis, viral hepatitis, cholestasis, steatohepatitis, alcoholic/non-alcoholic liver disease and drug-induced liver injury (DILI) [1, 2].

KCs exert such effects by both direct cell-cell contact with hepatocytes as well as release of a variety of inflammatory cytokines, growth factors and reactive oxygen species upon activation [3].

These interactions, especially under inflammatory conditions, are not recapitulated in mono-cultures using only primary human hepatocytes (pHeps), which are the gold standard for in vitro liver models. The lack of nonparenchymal cells, including KCs in pHeps mono-cultures, is a likely cause for suboptimal performance of in vitro models for liver disease modeling [4] and detection of hepatotoxicity [5, 6]. So far, models involving KCs have deployed cells from animal origin [7-9]. To avoid interspecies variability, it is important to study the effects of KCs from a human origin.

Applications of human KCs are hampered by donor availability, low yield and/or purity and tedious isolation procedures for primary adult human KCs (pKCs) [10, 11]. Once isolated/purified, pKCs cannot maintain their functions over extended periods nor be expanded in culture to produce more cells. The cost of pKCs remains a challenge: currently USD 1075 to USD 3900 per million pKCs which are supplied by a handful of commercial companies.

In order to replace pKCs, the alternative cells should be renewable, liver-specific and mature. Human induced pluripotent stem cells (iPSC)-derived cell source could be a valuable alternative; however, iPSC-derived cells often exhibit fetal-like rather than mature properties [12-16]. In addition, there is no successful protocol to generate KCs from iPSCs (iKCs) so far, possibly due to the long-lasting dogma that KCs originate from bone marrow-derived blood-circulating monocytes (BMDMs) [17, 18].

Contrasting studies since 2012 have demonstrated that KCs are established during embryonic development, independent of BMDMs [19-21]. Primitive macrophages (Mφ) generated in the yolk sac from early erythro-myeloid progenitors proliferate and differentiate into liver-specific macrophages, i.e. KCs upon receiving hepatic cues in the liver in a MYB-independent manner [21, 22]. Recent studies have shown that iPSC-derived Mφ share ontogeny with MYB-independent tissue-resident Mφ [23].

### SUMMARY

According to a 1^{st} aspect of the present invention, we provide a method of producing an iPSC-derived Kupffer Cell (iKC). The method may comprise providing a macrophage precursor (preMφ). The macrophage precursor may be derived from an induced pluripotent stem cell (iPSC).

The method may comprise culturing the macrophage precursor (preMφ) in the presence of a hepatic cue. The method may comprise obtaining an iPSC-derived Kupffer Cell (iKC) therefrom.

The iPSC-derived Kupffer Cell (iKC) may display a biological property of a primary Kupffer cell. The primary Kupffer cell may comprise a primary adult human KC (pKC).

The hepatic cue may comprise exposure to primary human hepatocyte conditioned media (PHCM). The macrophage precursor may be cultured in primary human hepatocyte conditioned media (PHCM). The culture may take place in Advanced DMEM.

The biological property may comprise expression of a macrophage marker. The biological property may comprise phagocytosis. The biological property may comprise release of an inflammatory cytokine upon activation. The biological property may comprise release of an growth factor upon activation. The biological property may comprise release of an oxygen species upon activation. The biological property may comprise secretion of IL-6 and TNFα upon stimulation. The stimulation may comprise exposure to LPS.

The biological activity may comprise expression of a macrophage marker.

The macrophage marker may comprise CD11 (GenBank Accession Number NM_000632.3). The macrophage marker may comprise CD14 (GenBank Accession Number NM_001174105.1). The macrophage marker may comprise CD68 (GenBank Accession Number NM_001251.2). The macrophage marker may comprise CD163 (GenBank Accession Number NM_203416.3). The macrophage marker may comprise CD32 (GenBank Accession Number NM_001136219.1). The macrophage marker may comprise CLEC-4F (GenBank Accession Number NM_173535.2). The macrophage marker may comprise ID1 (GenBank Accession Number NM_181353.2). The macrophage marker may comprise ID3 (GenBank Accession Number NM_002167.4).

The macrophage precursor (preMφ) may be derived from an induced pluripotent stem cell (iPSC) by culturing the induced pluripotent stem cell (iPSC) to generate an embryoid body (EB). The embryoid body (EB) may be cultured to generate a macrophage precursor (preMφ) cell.

Step (a) may comprise exposure to bone morphogenetic protein-4 (BMP-4, GenBank Accession Number Q53XC5). The BMP-4 may be present at 50 ng/mL. It may comprise exposure to vascular endothelial growth factor (VEGF, GenBank Accession Number NP_001165097). The VEGF may be present at 50 ng/mL. It may comprise exposure to stem cell factor (SCF, GenBank Accession Number P21583.1). The SCF may be present at 20 ng/mL. It may comprise exposure to ROCK Inhibitor. The ROCK Inhibitor may be present at 10 μM.

Step (a) may comprise exposure to a medium containing each of these, at the stated concentrations. Step (a) may comprise culture in such a medium.

Step (b) may comprise exposure to macrophage colony stimulating factor (M-CSF, GenBank Accession Number P09603). The M-CSF may be present at 100 ng/mL. It may comprise exposure to Interleukin-3 (IL-3, GenBank Accession Number AAC08706). The IL-3 may be present at 25 ng/mL. It may comprise exposure to glutamax. The glutamax may be present at 2 mM. It may comprise exposure to β-mercaptoethanol. The β-mercaptoethanol may be present at 0.055 mM.

Step (b) may comprise exposure to a medium containing each of these, at the stated concentrations. Step (b) may comprise culture in such a medium.

The induced pluripotent stem cell (iPSC) may comprise a MYB-independent iPSC.

There is provided, according to a 2^{nd} aspect of the present invention, an iPSC-derived Kupffer Cell (iKC) obtainable from a method according to the 1^{st} aspect of the invention.

We provide, according to a 3^{rd} aspect of the present invention, a combination of a iPSC-derived Kupffer Cell (iKC) as described with a hepatocyte. The combination may comprise a co-culture. The hepatocyte may comprise a primary human hepatocyte (pHEP). The hepatocyte may comprise an iPSC-derived hepatocyte (iHep). The iPSC-derived Kupffer Cell (iKC) and the hepatocyte may be donor matched. They may be derived from the same stem cell source.

As a 4^{th} aspect of the present invention, there is provided use of such a iPSC-derived Kupffer Cell (iKC) or such a combination or co-culture in a method for determining the hepatotoxicity of a drug. The drug may comprise an inflammation-associated drug. It may comprise Acetaminophen. The drug may comprise Trovafloxacin. It may comprise Chlorpromazine.

We provide, according to a 5^{th} aspect of the present invention, use of such an iPSC-derived Kupffer Cell (iKC) or such a combination or co-culture as a model for a disease or condition.

The disease or condition may comprise liver injury. The disease or condition may comprise drug-induced liver injury (DILI). The disease or condition may comprise liver disease. The disease or condition may comprise steatohepatitis. The disease or condition may comprise cholestasis. The disease or condition may comprise liver fibrosis. The disease or condition may comprise viral hepatitis.

The present invention, in a 6^{th} aspect, provides use of such an iPSC-derived Kupffer Cell (iKC) in the preparation of a medicament for the treatment or prevention of a disease or condition. The disease or condition may comprise a liver disease or condition.

The disease or condition may comprise liver injury. The disease or condition may comprise drug-induced liver injury (DILI). The disease or condition may comprise liver disease. The disease or condition may comprise steatohepatitis. The disease or condition may comprise cholestasis. The disease or condition may comprise liver fibrosis. The disease or condition may comprise viral hepatitis.

In a 7^{th} aspect of the present invention, there is provided a method of treatment or prevention of a disease or condition. The method may comprise administering or transplanting an iPSC-derived Kupffer Cell (iKC) as described to a patient in need of such treatment. The disease or condition may comprise a liver disease or condition.

The disease or condition may comprise liver injury. The disease or condition may comprise drug-induced liver injury (DILI). The disease or condition may comprise liver disease. The disease or condition may comprise steatohepatitis. The disease or condition may comprise cholestasis. The disease or condition may comprise liver fibrosis. The disease or condition may comprise viral hepatitis.

The practice of this invention will employ, unless otherwise indicated, conventional techniques of chemistry, molecular biology, microbiology, recombinant DNA and immunology, which are within the capabilities of a person of ordinary skill in the art. Such techniques are explained in the literature. See, for example, J. Sambrook, E. F. Fritsch, and T. Maniatis, 1989, Molecular Cloning: A Laboratory Manual, Second Edition, Books 1-3, Cold Spring Harbor Laboratory Press; Ausubel, F. M. et al. (1995 and periodic supplements; Current Protocols in Molecular Biology, ch. 9, 13, and 16, John Wiley & Sons, New York, N.Y.); B. Roe, J. Crabtree, and A. Kahn, 1996, DNA Isolation and Sequencing: Essential Techniques, John Wiley & Sons; J. M. Polak and James O′D. McGee, 1990, In Situ Hybridization: Principles and Practice; Oxford University Press; M. J. Gait (Editor), 1984, Oligonucleotide Synthesis: A Practical Approach, Irl Press; D. M. J. Lilley and J. E. Dahlberg, 1992, Methods of Enzymology: DNA Structure Part A: Synthesis and Physical Analysis of DNA Methods in Enzymology, Academic Press; Using Antibodies: A Laboratory Manual: Portable Protocol NO. I by Edward Harlow, David Lane, Ed Harlow (1999, Cold Spring Harbor Laboratory Press, ISBN 0-87969-544-7); Antibodies: A Laboratory Manual by Ed Harlow (Editor), David Lane (Editor) (1988, Cold Spring Harbor Laboratory Press, ISBN 0-87969-314-2), 1855. Handbook of Drug Screening, edited by Ramakrishna Seethala, Prabhavathi B. Fernandes (2001, New York, N.Y., Marcel Dekker, ISBN 0-8247-0562-9); and Lab Ref A Handbook of Recipes, Reagents, and Other Reference Tools for Use at the Bench, Edited Jane Roskams and Linda Rodgers, 2002, Cold Spring Harbor Laboratory, ISBN 0-87969-630-3. Each of these general texts is herein incorporated by reference.

### BRIEF DESCRIPTION OF THE FIGURES

FIGS. 1A to 1E are drawings showing differentiation of iKCs from iPSCs.

FIG. 1A is a drawing showing schematics and generation of EBs and macrophage precursors (preMφ) from iPSCs. EBs formed within 2 days and were allowed to grow till day 4, following which they were collected and grown in differentiation media for generation of preMφ. preMφ were then differentiated into kupffer cells (iKCs). Scale bar: 200 μm.

FIG. 1B is a drawing showing marker gene expression in precursors of iKCs: iPSCs (light grey bars) and preMφ (dark grey bars). Error bars represent s.e.m, n=3.

FIG. 1C to 1E are drawings showing phase contrast images of iKCs (FIG. 1C) in comparison with primary human kupffer cells, pKCs (FIG. 1D) and non-liver macrophages (NL-Mφ) (FIG. 1E). Scale bar: 50 EBs: embryoid bodies.

FIG. 2A to 2H are drawings showing gene marker expression of iKCs.

FIG. 2A is a drawing showing heat map analyses showing raw expression levels (log 2) of macrophage markers.

FIGS. 2B to 2F are drawings showing heat map showing genes belonging to pathways known to be involved in KC function: cytokine and inflammatory response (B), complement and coagulation cascade (FIG. 2C), intrinsic pathway for apoptosis (FIG. 2D), pattern recognition receptors (FIG. 2E) and Inhibitor of DNA binding proteins (ID) signaling (FIG. 2F). Freshly thawed pKCs from three independent donors were used. Three independent batches of differentiation were used for iKCs. Samples were collected at the end of the differentiation period (day 7). * indicate genes that are upregulated in pKCs and # indicate genes that are upregulated in iKCs by at least 2 fold (p<0.05).

FIG. 2G is a drawing showing gene expression analysis showing expression of macrophage markers in iKCs compared to pKCs at the end of the differentiation period (day 7).

FIG. 2H is a drawing showing gene expression of KC-specific markers in iKCs at different time points doing the seven day differentiation period (grey shaded bars). Single solid lines represent p<0.05 and double solid lines represent p<0.01 (two-tailed paired t-test). Error bars represent s.e.m, n=3. UD: Undetectable.

FIGS. 3A and 3B are drawings showing protein marker expression of iKCs.

FIG. 3A is a drawing showing protein expression of macrophage markers detected by immunofluorescence in iKCs and pKCs. Cell nuclei were stained with DAPI. Scale bar: 50 μm. Percentages indicate the proportion of positively stained cells calculated using number of fluorescently labelled cells and total number of cells (DAPI-stained).

FIG. 3B is a drawing showing representative histograms of flow cytometric analysis to determine marker expression of CD68, CD163 and CLEC-4F in iKCs. Positive gates were defined by unstained samples and isotype control. Percentages indicate proportion of positive cells from three independent differentiations.

FIG. 4A to 4E are drawings showing marker differences between iKCs and NL-Mφ.

FIG. 4A is a drawing showing principle component analysis of transcriptional profiles and (FIG. 4B) dendrogram showing hierarchical clustering of iKCs compared with pKCs, iPSCs, monocytes and other non-liver-resident Mφ.

FIG. 4C is a drawing of heat map showing expression level of genes in iKCs and NL-Mφ which have been reported to show a differential expression between liver-resident KCs and Mφ resident in other non-liver tissues. Average of three independent batches of NL-Mφ and iKCs are shown. BMDM: bone marrow-derived blood-circulating monocytes, (FIG. 4D to FIG. 4E) Expression of CLEC-4F at gene and protein level. UD: Undetectable. Scale bar: 30 μm

FIGS. 5A to 5E are drawings showing functional similarities between pKCs and iKCs and differences to NL-Mφ.

FIG. 5A is a drawing showing phagocytosis of fluorescent beads by iKCs, pKCs and NL-Mφ. Scale bar: 30 μm. At least ten images from each of three independent experiments were analysed and representative images are shown. Grey shading of cells represent CD163 staining and bright white dots (pointed by white arrows) show the phagocytosed beads.

FIG. 5B and FIG. 5C are drawings showing quantification of phagocytosis in iKCs, pKCs and NL-Mφ. Cells were stained with CD163 to quantify the number of cells in each field of image. Co-localization of fluorescent beads with CD163 staining was used to quantify percentage of phagocytising cells. Beads uptaken by each cell were counted and normalized to the number of cells in each field of image to obtain average number of beads per cell. Confocal microscopy was used to ensure that only beads that were uptaken were counted. Solid black lines represent p<0.01 (two-tailed paired t-test). Error bars represent s.e.m, n=3.

FIG. 5D is a drawing showing phase contrast images showing morphology of iKCs (upper panel) in comparison NL-Mφ (lower panel) with and without LPS treatment. Scale bar: 100 μm

FIG. 5E is a drawing showing IL-6 and TNFα production in iKCs in comparison with pKCs, NL-Mφ and pHeps before and after LPS stimulation. Dashed lines represent fold differences between cytokine production before and after LPS treatment (folds are indicated on top of the dashed lines). Solid lines represent p<0.05. Error bars represent s.e.m, n=3. UD: Undetectable, LPS: Lipopolysaccharide, IL-6: Interleukin-6, TNFα: Tumor necrosis factor alpha, pHeps: Primary human hepatocytes.

FIGS. 6A to 6K are drawings showing establishment of hepatocytes and KCs co-culture model

FIG. 6A is a drawing showing Left panel: schematics of co-culture set up and right panel: image of co-culture showing pHeps (albumin positive) and pKCs (CD163 positive); Scale bar: 50 μm.

FIG. 6B is a drawing showing basal activity of CYP1A2, CYP3A4 and CYP2B6 in mono-culture of pHeps and co-culture of pHeps and pKCs in Medium A (commercially recommended Advanced DMEM based medium) and William's E Medium without dexamethasone (Dex) at day 5. Solid lines represent p<0.05. Error bars represent s.e.m, n=3.

FIG. 6C is a drawing showing gene expression of macrophage markers (left panel) and KC-specific markers (right panel) in pKCs at day 5 when co-cultured with pHeps in William's medium without Dex. Expression levels are presented relative to gene expression of freshly thawed pKCs.

FIG. 6D is a drawing showing CYP3A4 and CYP2C19 gene expression (left panel) and albumin production (right panel) in co-culture of pHeps-pKCs (light grey bars) and pHeps-iKCs (dark grey bars) in William's E Medium without Dex at day 5.

FIG. 6E is a drawing showing gene expression of macrophage markers (left panel) and KC-specific markers (right panel) in iKCs at day 5 when co-cultured with pHeps in William's medium without Dex. Expression levels are presented relative to gene expression of freshly thawed pKCs.

FIG. 6F is a drawing showing the cell viability of pHeps in co-culture with pKCs, iKCs and NL-Mφ was assessed by Alamar Blue® assay after exposure to different concentrations of test compounds (FIG. 6F to FIG. 6H: APAP) and (FIG. 6I to FIG. 6K: Trovafloxacin) and compared to mono-culture of hepatocytes in the presence and absence of LPS. Cell viability is expressed as a percentage of cells treated with solvent alone. Horizontal solid lines indicate 50% cell viability. Dashed curves represent cultures which were not treated with LPS. Solid curves represent cultures treated with LPS. Black curves represent mono-culture of pHeps and grey curves represent co-cultures. Error bars represent s.e.m, n=3. * indicates statistically significant differences between LPS-activated mono-culture and co-culture; # indicate statistically significant differences between non-activated mono-culture and co-culture (p<0.05). APAP: Acetaminophen.

FIGS. 7A to 7I are drawings showing application of iKCs to a donor-matched inflammatory model for detecting hepatotoxicity and modelling cholestatic disease.

FIG. 7A is a drawing showing immune response elicited by donor-mismatched immune cells as shown by production of cytokines, IL-6 and TNFα in mono-culture of pHeps and pKCs when stimulated with LPS, and in co-culture of iHeps and iKCs (donor-matched co-culture; hepatocytes and KCs derived from the same iPSC source) and pHeps and pKCs (donor-mismatched co-culture; different donors) without any endotoxin stimulation. Error bars represent s.e.m, n≥3. Single solid lines represent p<0.05 and double solid lines represent p<0.01. UD: Undetectable

FIG. 7B is a drawing showing an image of co-culture showing human iPSC-hepatocytes (iHeps) (albumin positive) and iKCs (CD163 positive); Scale bar: 50 μm.

FIG. 7C is a drawing showing gene expression of hepatic markers in iHeps at day 5 when co-cultured with iKCs in William's medium without Dex. AFP: alpha-fetoprotein, ALB: albumin, AAT: Alpha 1-antitrypsin and ASGPR: asialoglycoprotein receptor

FIG. 7D is a drawing showing gene expression of Mφ markers (left panel) and KC-specific markers (right panel) in iKCs at day 5 when co-cultured with iHeps in William's medium without Dex.

FIG. 7E and FIG. 7F are drawings showing the cell viability of iHeps in co-culture with iKCs and NL-Mφ was assessed by Alamar Blue® assay after exposure to different concentrations of APAP and compared to mono-cultures in the presence and absence of LPS. Cell viability is expressed as a percentage of cells treated with solvent alone. Horizontal solid lines across indicate 50% cell viability. Dashed lines represent cultures which were not treated with LPS. Solid lines represent cultures treated with LPS. Black lines represent mono-culture of hepatocytes and grey lines represent co-cultures. Error bars represent s.e.m, n≥3. Hep: hepatocytes.

FIG. 7G is a drawing showing IL-6 and TNFα production in iHep/iKCs co-culture and iHeps mono-culture when treated with LPS and paradigm cholestatic drug chlorpromazine (CPZ, 10 μM). Levels are expressed as fold change compared to untreated control

FIG. 7H is a drawing showing accumulation of FDA in iHeps-iKCs co-culture upon treatment compared to untreated control (left panel); Scale bar: 50 μm. (I) Quantification of bile acid accumulation using ImageJ. Solid line represents p<0.05 (J) Gene expression of BSEP, MDR1 and MRP1 in co-culture, compared to mono-culture upon CPZ treatment. Expression levels are presented as fold change to untreated control. Error bars represent s.e.m, n≥3. Solid line represents p<0.05. BSEP: bile salt export pump, MDR1: multidrug resistance, MRP1: multidrug resistance associated protein.

FIGS. 8A to 8E are drawings showing phase contrast images showing iKCs viability, attachment and density on different media and extracellular matrix configurations.

preMφ were differentiated for seven days in primary human hepatocyte conditioned media (PHCM) alone (FIG. 8A), PHCM and X-VIVO media (FIG. 8B) PHCM and RPMI-1640 and 10% serum (FIG. 8C), PHCM media and X-VIVO and 10% serum (FIG. 8D) and PHCM media and Advanced DMEM. Scale bar: 100 μm.

FIGS. 9A to 9D are drawings showing establishment of hepatocytes and KCs co-culture model and treatment with Levofloxacin.

FIG. 9A is a drawing showing basal activity of CYP1A2, CYP3A4 and CYP2B6 of pHeps mono-culture in William's E Medium with (light grey bars) and without Dex (dark grey bars) at day 5.

FIG. 9B is a drawing showing CYP3A4 and CYP2C19 gene expression and albumin production in mono-culture of pHeps, co-culture of pHeps-pKCs and pHeps-iKCs in William's E Medium without Dex at day 1-5. Error bars represent s.e.m, n=3.

FIG. 9C and FIG. 9D are drawings showing treatment of pHeps-pKCs co-culture and iHeps-iKCs with non-hepatotoxic compound, Levofloxacin. The cell viability of pHeps in co-culture with pKCs (FIG. 9C) and iHeps in co-culture with iKCs (FIG. 9D) was assessed by Alamar Blue® assay after exposure to different concentrations of Levofloxacin in the presence and absence of LPS. Respective mono-cultures were used as controls. Cell viability is expressed as a percentage of cells treated with solvent alone. Horizontal solid lines across indicate 50% cell viability. Dashed lines represent cultures which were not treated with LPS. Solid lines represent cultures treated with LPS. Black lines represent mono-culture of hepatocytes and grey lines represent co-cultures.

### DETAILED DESCRIPTION

Liver macrophages, Kupffer cells (KCs), play a critical role in drug-induced liver injury (DILI) and liver diseases including cholestasis, liver fibrosis and viral hepatitis. Application of KCs in in vitro models of DILI and liver diseases is hindered due to limited source of human KCs.

In vivo, KCs originate from MYB-independent macrophage progenitors, which differentiate into liver-specific macrophages in response to hepatic cues in the liver.

We aimed to generate iPSC-derived Mφ precursors (preMφ) and provide them with hepatic cues in vitro to drive them towards liver-specific iKCs. We hypothesized that this method would allow generation of a renewable source of liver-specific and mature iKCs, which could be used in various applications.

Here, we recapitulated KCs ontogeny by differentiation of MYB-independent iPSCs to macrophage-precursors and exposing them to hepatic cues to generate iPSC-derived KCs (iKCs).

Molecular and functional assays demonstrated that iKCs are similar to pKCs but different from other non-liver Mφ (NL-Mφ), indicating that they are mature and liver specific.

iKCs expressed macrophage markers (CD11/CD14/CD68/CD163/CD32) at 0.3-5 folds of primary adult human KCs (pKCs) and KC-specific CLEC-4F, ID1 and ID3. iKCs phagocytosed and secreted IL-6 and TNFα upon stimulation at levels similar to pKCs but different from non-liver macrophages. Hepatocyte-iKCs co-culture model was more sensitive in detecting hepatotoxicity induced by inflammation-associated drugs, Acetaminophen and Trovafloxacin, and Chlorpromazine-induced cholestasis when compared to hepatocytes alone. Overall, iKCs were mature, liver-specific and functional.

iKCs were co-cultured with hepatocytes generated from the same iPSC donor to establish a donor-matched co-culture model which could model inflammation-associated hepatotoxicity and cholestatic disease.

Donor-matched iKCs and iPSC-hepatocyte co-culture exhibited minimal non-specific background response compared to donor-mismatched counterpart. iKCs offer a mature renewable human cell source for liver-specific macrophages, useful in developing in vitro model to study DILI and liver diseases such as cholestasis.

Production of iPSC-Derived Kupffer Cells (iKCs)

We disclose a method of producing a Kupffer cell from an induced pluripotent stem cell (iPSC). We term such a Kupffer cell an iPSC-derived Kupffer Cell (iKC).

Our method involves providing a macrophage precursor cell (preMφ). Such a macrophage precursor cell may be derived from an induced pluripotent stem cell (iPSC).

The method further comprises exposing the macrophage precursor cell (preMφ) to one or more cues which bias its differentiation into a Kupffer cell. The cue may comprise a hepatic cue and the macrophage precursor cell (preMφ) may be cultured in the presence of such a hepatic cue.

The iPSC-derived Kupffer cell may have a biological property, such as a biological activity of a Kupffer cell.

### Kupffer Cell

Kupffer cells are described in detail in Dixon L J, Barnes M, Tang H, Pritchard M T, Nagy L E. Kupffer cells in the liver. Compr Physiol. 2013; 3(2):785-797. doi:10.1002/cphy.c120026 and Bilzer M, Roggel F, Gerbes A L. Role of Kupffer cells in host defense and liver disease. Liver Int. 2006 December; 26(10):1175-86.

Kupffer cells, also known as stellate macrophages and Kupffer-Browicz cells, are specialized macrophages located in the liver, lining the walls of the sinusoids. They form part of the mononuclear phagocyte system.

These cells were first observed by Karl Wilhelm von Kupffer in 1876, who called them “Sternzellen” (star cells or hepatic stellate cell) but thought, inaccurately, that they were an integral part of the endothelium of the liver blood vessels and that they originated from it. In 1898, after several years of research, Tadeusz Browicz identified them, correctly, as macrophages.

Kupffer cell development begins in the yolk sac where they differentiate into fetal macrophages. Once they enter the blood stream, they migrate to the fetal liver where they stay. There they complete their differentiation into Kupffer cells.

Apart from clearing any bacteria, red blood cells are also broken down by phagocytic action, where the hemoglobin molecule is split. The globin chains are re-used, while the iron-containing portion, heme, is further broken down into iron, which is re-used, and bilirubin, which is conjugated to glucuronic acid within hepatocytes and secreted into the bile.

Helmy et al. identified a receptor present in Kupffer cells, the complement receptor of the immunoglobulin family (CRIg). Mice without CRIg could not clear complement system-coated pathogens. CRIg is conserved in mice and humans and is a critical component of the innate immune system.

Kupffer cell activation is responsible for early ethanol-induced liver injury, common in chronic alcoholics. Chronic alcoholism and liver injury deal with a two hit system. The second hit is characterized by an activation of the Toll-like receptor 4 (TLR4) and CD14, receptors on the Kupffer cell that internalize endotoxin (lipopolysaccharide or LPS). This activates the transcription of pro-inflammatory cytokines (Tumor necrosis factor-alpha or TNFα) and production of superoxides (a pro-oxidant). TNFα will then enter the stellate cell in the liver, leading to collagen synthesis and fibrosis. Fibrosis will eventually cause cirrhosis, or loss of function of the liver.

Generation of Macrophage Precursor Cell (preMΦ) from Induced Pluripotent Stem Cell (iPSC)

Methods of producing macrophage precursors from iPSCs are known in the art and are described for example in:

Buchrieser J, James W, Moore M D. Human Induced Pluripotent Stem Cell-Derived Macrophages Share Ontogeny with MYB-Independent Tissue-Resident Macrophages. Stem Cell Reports. 2017; 8(2):334-345. doi:10.1016/j.stemcr.2016.12.020

Hale C, Yeung A, Goulding D, et al. Induced pluripotent stem cell derived macrophages as a cellular system to study salmonella and other pathogens. PLoS One. 2015; 10(5):e0124307. Published 2015 May 6. doi:10.1371/journal.pone.0124307

Mukherjee C., Hale C., Mukhopadhyay S. (2018) A Simple Multistep Protocol for Differentiating Human Induced Pluripotent Stem Cells into Functional Macrophages. In: Rousselet G. (eds) Macrophages. Methods in Molecular Biology, vol 1784. Humana Press, New York, N.Y.

Kazuyuki Takata, Tatsuya Kozaki, Christopher Zhe Wei Lee, Morgane Sonia Thion, Masayuki Otsuka, Shawn Lim, Kagistia Hana Utami, Kerem Fidan, Dong Shin Park, Benoit Malleret, Svetoslav Chakarov, Peter See, Donovan Low, Gillian Low, Marta Garcia-Miralles, Ruizhu Zeng, Jinqiu Zhang, Chi Ching Goh, Ahmet Gul, Sandra Hubert, Bernett Lee, Jinmiao Chen, Ivy Low, Nurhidaya Binte Shadan, Josephine Lum, Tay Seok Wei, Esther Mok, Shohei Kawanishi, Yoshihisa Kitamura, Anis Larbi, Michael Poidinger, Laurent Renia, Lai Guan Ng, Yochai Wolf, Steffen Jung, Tamer Onder, Evan Newell, Tara Huber, Eishi Ashihara, Sonia Garel, Mahmoud A. Pouladi, Florent Ginhoux. Induced-Pluripotent-Stem-Cell-Derived Primitive Macrophages Provide a Platform for Modeling Tissue Resident Macrophage Differentiation and Function. Immunity, Volume 47, Issue 1, 2017, Pages 183-198.e6, ISSN 1074-7613,

The method may involve a first step of generating an embryoid body (EB) from an Induced Pluripotent Cell (iPSC) and a second step of generating a macrophage precursor cell (preMφ) from the embryoid body (EB).

The first step may include exposing the iPSC to one or more factors, for example in culture. The iPSC may be exposed to bone morphogenetic protein-4 (BMP-4, GenBank Accession Number Q53XC5) preferably at 50 ng/mL The iPSC may be exposed to vascular endothelial growth factor (VEGF, GenBank Accession Number NP_001165097) preferably at 50 ng/mL. The iPSC may be exposed to stem cell factor (SCF, GenBank Accession Number P21583.1) preferably at 20 ng/mL. The iPSC may be exposed to ROCK Inhibitor preferably at 10 μM. The iPSC may be exposed to all of these factors simultaneously.

The second step may include exposing the EB to a factor, for example in culture. The EB may be exposed to macrophage colony stimulating factor (M-CSF, GenBank Accession Number P09603) preferably at 100 ng/mL. The EB may be exposed to Interleukin-3 (IL-3, GenBank Accession Number AAC08706) preferably at 25 ng/mL. The EB may be exposed to glutamax preferably at 2 mM. The EB may be exposed to β-mercaptoethanol preferably at 0.055 mM. The EB may be exposed to one or more, such as all of these factors simultaneously.

The EB may be exposed to the factor or factors in a medium such as X-VIVO™15 media (Lonza, Basel, Switzerland).

### Example Protocol for Generation of Macrophage Precursor Cell (preMφ) from Induced Pluripotent Stem Cell (iPSC)

An example protocol adapted from and Wilgenburg et al. [26] follows:

iPSCs are harvested using TrypLE™, centrifuged and the cell pellet was resuspended in stem cell maintenance media mTeSR™1, supplemented with 50 ng/mL bone morphogenetic protein-4 (BMP-4, GenBank Accession Number Q53XC5), 50 ng/mL vascular endothelial growth factor (VEGF, GenBank Accession Number NP_001165097), 20 ng/mL stem cell factor (SCF, GenBank Accession Number P21583.1) and 10 μM ROCK Inhibitor (Calbiochem, Billerica, Mass., USA).

The cells are seeded at a density of 12,000 cells/well into a round-bottom, low adherence, 96-well plate, which is centrifuged and incubated at 37° C. in a 5% CO₂ atmosphere for 4 days before harvesting the embryoid bodies (EBs).

75% media change is performed on the second day.

At day 4, 12 EBs are harvested and transferred into each well of a 6-well plate and cultured in X-VIVO™15 media (Lonza, Basel, Switzerland), supplemented with 100 ng/mL macrophage colony stimulating factor (M-CSF, GenBank Accession Number P09603), 25 ng/mL Interleukin-3 (IL-3, GenBank Accession Number AAC08706), 2 mM glutamax, 100 U/mL penicillin and 100 mg/mL streptomycin and 0.055 mM β-mercaptoethanol (Sigma-Aldrich, Singapore).

Two-thirds of the media is changed every five to seven days. preMφ are generated from the EBs in 3 to 4 weeks.

Suspended preMφ are collected from the media weekly. They may be used for further differentiation.

Induced Pluripotent Stem Cell (iPSC)

Induced Pluripotent Stem Cells (iPSC) are morphologically similar to human embryonic stem cells, express typical human ESC-specific cell surface antigens and genes, differentiate into multiple lineages in vitro, and form teratomas containing differentiated derivatives of all three primary germ layers when injected into immunocompromised mice.

Human iPS cells are derived from somatic cells and are typically produced by expression of Oct-3/4, Sox-2, c-Myc, and Klf-4 or by Oct-3/4, Sox-2, Nanog, and Lin28.

Methods of producing induced pluripotent stem cells are known in the art, and are described for example in WO 2016/120493, EP2128245, U.S. Pat. No. 7,682,828, etc.

The term “pluripotent” or “pluripotency” as used herein refers to cells with the ability to give rise to progeny that can undergo differentiation, under the appropriate conditions, into cell types that collectively demonstrate characteristics associated with cell lineages from all of the three germinal layers (endoderm, mesoderm, and ectoderm). Pluripotent stem cells can contribute to many or all tissues of a prenatal, postnatal or adult animal.

A standard art-accepted test, such as the ability to form a teratoma in 8-12 week old SCID mice, can be used to establish the pluripotency of a cell population, however identification of various pluripotent stem cell characteristics can also be used to detect pluripotent cells. Cell pluripotency is a continuum, ranging from the completely pluripotent cell that can form every cell of the embryo proper, e.g., embyronic stem cells and iPSCs, to the incompletely or partially pluripotent cell that can form cells of all three germ layers but that may not exhibit all the characteristics of completely pluripotent cells, such as, for example, germline transmission or the ability to generate a whole organism. In particular embodiments of the invention, the pluripotency of a cell is increased from an incompletely or partially pluripotent cell to a more pluripotent cell or, in certain embodiments, a completely pluripotent cell.

Pluripotency can be assessed, for example, by teratoma formation, germ-line transmission, and tetraploid embryo complementation. In some embodiments, expression of pluripotency genes or pluripotency markers as discussed elsewhere herein, can be used to assess the pluripotency of a cell.

“Pluripotent stem cell characteristics” refer to characteristics of a cell that distinguish pluripotent stem cells from other cells. The ability to give rise to progeny that can undergo differentiation, under the appropriate conditions, into cell types that collectively demonstrate characteristics associated with cell lineages from all of the three germinal layers (endoderm, mesoderm, and ectoderm) is a pluripotent stem cell characteristic.

Expression or non-expression of certain combinations of molecular markers are also pluripotent stem cell characteristics. For example, human pluripotent stem cells express at least some, and optionally all, of the markers from the following non-limiting list: SSEA-3, SSEA-4, TRA-1-60, TRA-1-81, TRA-2-49/6E, ALP, Sox2, E-cadherin, UTF-1, Oct4, Rexl, and Nanog. Cell morphologies associated with pluripotent stem cells are also pluripotent stem cell characteristics. A “somatic cell” as used herein refers to differentiated, or partially differentiated cells relative to embryonic stem cells. Thus, the term includes, e.g., cells such as fibroblasts that are derived from embryonice stem cells, but are differentiated.

The term “embryonic stem cell” is used to refer to the pluripotent stem cells of the inner cell mass of the embryonic blastocyst (see U.S. Pat. Nos. 5,843,780, 6,200,806). The distinguishing characteristics of an embryonic stem cell define an embryonic stem cell phenotype. Accordingly, a cell has the phenotype of an embryonic stem cell if it possesses one or more of the unique characteristics of an embryonic stem cell such that that cell can be distinguished from other cells. Illustrative distinguishing embryonic stem cell characteristics include, without limitation, gene expression profile, proliferative capacity, differentiation capacity, normal karyotype, responsiveness to particular culture conditions, and the like.

A “cancer stem cell” as used herein refers to self-renewing and pluripotent cancer cells (see, e.g., U.S. Pat. No. 6,984,522). Such cells can be obtained from any tumor source including primary or any metastatic tumor site, lymph nodes, ascites fluids, or blood. Cancer stem cells are identified by virtue of their functional characteristics that include, without limitation, the ability to repopulate new tumors in serial transplants, and ability to give rise to the functional and phenotypic cellular heterogeneity of the original tumor.

Macrophage Precursor Cell (preMΦ)

Generation of iPSC-Derived Kupffer Cell (iKC) from Macrophage Precursor Cell (preMφ)

The macrophage precursor cell may be exposed to one or more hepatic cues to promote its maturation into an Kupffer Cell. For example, the macrophage precursor cell may be cultured in a cell medium comprising one or more such hepatic cues.

The hepatic cue may comprise one or more factors secreted by a hepatocyte. For this purpose, hepatocytes may be cultured in a cell culture medium and the soluble factors released by the hepatocytes may be collected in the form of medium conditioned by hepatocyte cell culture (a primary hepatocyte culture medium, PHCM).

This conditioned medium may be collected and added to the premacrophage culture medium to expose them to the cue.

The macrophage precursor cell may be exposed to a single factor or a combination of factors.

The cell culture medium may comprise Advanced DMEM. The cell medium may therefore be supplemented with conditioned medium from culture of hepatocytes, for example conditioned medium from culture of primary human hepatocytes (primary human hepatocyte conditioned medium—PHCM).

The cell culture medium may contain a supplement. The supplement may comprise any of the following, such as at the indicated concentrations:
2.5 mL Penicillin/Streptomycin (10,000 U/mL/(10,000 μg/mL) Final concentration: 0.5%
human recombinant insulin (6.25 μg/mL final concentration)
human transferrin (6.25 μg/mL final concentration)
selenous (6.25 μg/mL final concentration)
bovine serum albumin (BSA)—(1.25 g/mL)
linoleic acid (5.35 μg/mL final concentration)
HEPES, pH 7.4 (15 mM final concentration)
GlutaMAX™ 2 mM final concentration

More than one of the supplemental may be present. The supplements may be purchased commercially as a cocktail. For example, the supplements may be purchased from Invitrogen (as “cocktail b”) https://www.thermofisher.com/sg/en/home/references/protocols/drug-discovery/adme-tox-protocols/media-supplement-guide.html

### Hepatic Cue

According to the methods described here, a macrophage precursor (preMφ) cell is exposed to one or more hepatic cues to generate an iPSC-derived Kupffer cell. The macrophage precursor (preMφ) cell may be cultured in the presence of the or each hepatic cue.

Advanced DMEM The hepatic cue may comprise culture in Advanced DMEM. The culture medium may therefore comprise Advanced DMEM containing conditioned medium from another source, for example primary human hepatocyte conditioned media (PHCM).

Advanced DMEM may be obtained commercially, for example from Thermo Fisher Scientific (catalogue number 12491015).

Primary Human Hepatocyte Conditioned Medium (PHCM)

The conditioned cell culture medium such as a Primary Human Hepatocyte Conditioned Medium (PHCM) may be obtained by culturing a hepatocyte such as a human hepatocyte, a descendent thereof or a cell line derived therefrom in a cell culture medium; and isolating the cell culture medium.

Methods of culturing primary hepatocytes are well known in the art and are for example described in Shulman M, Nahmias Y. Long-term culture and coculture of primary rat and human hepatocytes. Methods Mol Biol. 2013; 945:287-302. doi:10.1007/978-1-62703-125-7_17.

The conditioned medium may be filtered or concentrated or both during, prior to or subsequent to use. For example, it may be filtered through a membrane, for example one with a size or molecular weight cut-off. It may be subject to tangential force filtration or ultrafiltration.

For example, filtration with a membrane of a suitable molecular weight or size cutoff, may be used.

The conditioned medium, optionally filtered or concentrated or both, may be subject to further separation means, such as column chromatography. For example, high performance liquid chromatography (HPLC) with various columns may be used. The columns may be size exclusion columns or binding columns.

iPSC-Derived Kupffer Cell (iKC)

The iPSC-derived Kupffer cell prepared according to the methods described here may exhibit a property of a Kupffer cell. The property of a Kupffer Cell may comprise a biological property, such as a biological activity.

The iPSC-derived Kupffer cell may exhibit any one or more of the biological activities of a Kupffer cell, such as a human Kupffer cell. The iPSC-derived Kupffer cell may for example have a diagnostic, therapeutic or restorative activity of a Kupffer cell.

The Kupffer cell may comprise a native Kupffer cell. The native Kupffer cell may comprise a Kupffer cell from a liver of an individual. The native Kupffer cell may comprise a primary Kupffer cell. The native Kupffer cell may comprise a primary adult human KC (pKC).

The Examples show that iPSC-derived Kupffer cells comprise biological activities of Kupffer cells and are capable of substituting for the Kupffer cells themselves. The biological property or biological activity of an iPSC-derived Kupffer cell may therefore correspond to a biological property or activity of a Kupffer cell.

The property may comprise a biological property such as a biological activity. Examples of biological activities of Kupffer cells include expression of a macrophage marker, phagocytosis, release of an inflammatory cytokine, growth factor or reactive oxygen species upon activation; and secretion of IL-6 and TNFα upon stimulation, preferably with LPS.

The iPSC-derived Kupffer cells may exhibit one or more such activities. The iPSC-derived Kupffer cells may display each of these activities.

### Expression of Macrophage Marker

The iPSC-derived Kupffer cell may exhibit a biological property of a Kupffer cell comprising expression of a macrophage marker such as a macrophage specific marker.

The macrophage marker may comprise CD11 (GenBank Accession Number NM_000632.3), CD14 (GenBank Accession Number NM_001174105.1), CD68 (GenBank Accession Number NM_001251.2), CD163 (GenBank Accession Number NM_203416.3) or CD32 (GenBank Accession Number NM_001136219.1). Alternatively, or in addition, the macrophage marker may comprise CLEC-4F (GenBank Accession Number NM_173535.2), ID1 (GenBank Accession Number NM_181353.2) or ID3 (GenBank Accession Number NM_002167.4).

Assays for expression of these markers are well known in the art.

### Phagocytosis

The iPSC-derived Kupffer cell may exhibit a biological property of a Kupffer cell such as phagocytosis.

Phagocytosis assays are known in the art and are for example described in Example 8.

The iPSC-derived Kupffer cell may exhibit release of an inflammatory cytokine, growth factor or reactive oxygen species upon activation. The iPSC-derived Kupffer cell may secrete IL-6 and TNFα upon stimulation, preferably with LPS. Such assays are known in the art and are described in the Examples, such as at Example 7.

Uses of iPSC-Derived Kupffer Cells

The iPSC-derived Kupffer cell may be used as a substitute for a Kupffer cell, as described above In particular, the iPSC-derived Kupffer cell may be used for any of the therapeutic purposes that Kupffer cells are currently being used, or in the future may be used.

It will be evident that the methods and compositions described here enable the production of Kupffer cells from iPSCs. Thus, any uses of Kupffer cells will equally attach to Kupffer cells derived from iPSCs.

iPSC-derived Kupffer cells produced by the methods and compositions described here may be used for, or for the preparation of a pharmaceutical composition for, the treatment of a disease or condition. Such disease or condition may comprise a a liver disease or condition, preferably selected from the group consisting of: liver injury, drug-induced liver injury (DILI), liver disease, steatohepatitis, cholestasis, liver fibrosis and viral hepatitis. Accordingly, iPSC-derived Kupffer cell may be used to treat such diseases.

iPSC-derived Kupffer cells such as those made according to the methods and compositions described here may be used for a variety of commercially important research, diagnostic, and therapeutic purposes.

The iPSC-derived Kupffer cells may in particular be used for the preparation of a pharmaceutical composition for the treatment of a disease or condition. Such a disease or condition may comprise a liver disease or condition, preferably selected from the group consisting of: liver injury, drug-induced liver injury (DILI), liver disease, steatohepatitis, cholestasis, liver fibrosis and viral hepatitis.

iPSC-derived Kupffer cells made by the methods and compositions described here have similar or identical properties to primary Kupffer cells. Therefore, the iPSC-derived Kupffer cells, may be used in any of the applications for which primary Kupffer cells are known to be used, or in which it is possible for them to be used.

Liver Injury

A liver injury, also known as liver laceration, is some form of trauma sustained to the liver. This can occur through either a blunt force such as a car accident, or a penetrating foreign object such as a knife. Liver injuries constitute 5% of all traumas, making it the most common abdominal injury.

Given its anterior position in the abdominal cavity and its large size, it is prone to gun shot wounds and stab wounds. Its firm location under the diaphragm also makes it especially prone to shearing forces. Common causes of this type of injury are blunt force mechanisms such as motor vehicle accidents, falls, and sports injuries. Typically these blunt forces dissipate through and around the structure of the liver and causes irreparable damage to the internal microarchitecture of the tissue. With increasing velocity of the impact, the internal damage of the liver tissue also exemplifies—even though the tissue itself is mechanically and micro-structurally isotropic. A large majority of people who sustain this injury also have another accompanying injury.

Drug-Induced Liver Injury (DILI)

The following description is abstracted from David S, Hamilton J P. Drug-induced Liver Injury. US Gastroenterol Hepatol Rev. 2010; 6:73-80.

Drug-induced liver injury (DILI) is common and nearly all classes of medications can cause liver disease.

Adverse drug reactions are an important cause of liver injury that may require discontinuation of the offending agent, hospitalization, or even liver transplantation.

Indeed, drug-induced hepatotoxicity is the most frequent cause of acute liver failure in US. Because the liver is responsible for concentrating and metabolizing a majority of medications, it is a prime target for medication-induced damage.

Among hepatotoxic drugs, acetaminophen (paracetamol) is the most often studied.

However, a broad range of different pharmacological agents can induce liver damage, including anesthetics, anticancer drugs, antibiotics, antituberculosis agents, antiretrovirals, and cardiac medications. In addition, a plethora of traditional medical therapies and herbal remedies may also be hepatotoxic.

DILI may be the result of direct toxicity from the administered drug or their metabolites, or injury may result from immune-mediated mechanisms.

Steatohepatitis

Steatohepatitis is a type of fatty liver disease, characterized by inflammation of the liver with concurrent fat accumulation in liver. Mere deposition of fat in the liver is termed steatosis, and together these constitute fatty liver changes.

There are two main types of fatty liver disease: alcohol-related fatty liver disease and non-alcoholic fatty liver disease (NAFLD). Risk factors for NAFLD include diabetes, obesity and metabolic syndrome.

hen inflammation is present it is referred to as alcoholic steatohepatitis and nonalcoholic steatohepatitis (NASH). Steatohepatitis of either cause may progress to cirrhosis, and NASH is now believed to be a frequent cause of unexplained cirrhosis (at least in Western societies). NASH is also associated with lysosomal acid lipase deficiency.

Cholestasis

Cholestasis is a condition where bile cannot flow from the liver to the duodenum. The two basic distinctions are an obstructive type of cholestasis where there is a mechanical blockage in the duct system that can occur from a gallstone or malignancy, and metabolic types of cholestasis which are disturbances in bile formation that can occur because of genetic defects or acquired as a side effect of many medications.

Liver Fibrosis

Cirrhosis, also known as liver cirrhosis or hepatic cirrhosis, is a condition in which the liver does not function properly due to long-term damage. This damage is characterized by the replacement of normal liver tissue by scar tissue. Typically, the disease develops slowly over months or years. Early on, there are often no symptoms. As the disease worsens, a person may become tired, weak, itchy, have swelling in the lower legs, develop yellow skin, bruise easily, have fluid build up in the abdomen, or develop spider-like blood vessels on the skin. The fluid build-up in the abdomen may become spontaneously infected. Other serious complications include hepatic encephalopathy, bleeding from dilated veins in the esophagus or dilated stomach veins, and liver cancer. Hepatic encephalopathy results in confusion and may lead to unconsciousness.

Cirrhosis is most commonly caused by alcohol, hepatitis B, hepatitis C, and non-alcoholic fatty liver disease. Typically, more than two or three alcoholic drinks per day over a number of years is required for alcoholic cirrhosis to occur. Non-alcoholic fatty liver disease has a number of causes, including being overweight, diabetes, high blood fats, and high blood pressure. A number of less common causes of cirrhosis include autoimmune hepatitis, primary biliary cholangitis, hemochromatosis, certain medications, and gallstones. Diagnosis is based on blood testing, medical imaging, and liver biopsy.

Some causes of cirrhosis, such as hepatitis B, can be prevented by vaccination. Treatment partly depends on the underlying cause, but the goal is often to prevent worsening and complications. Avoiding alcohol is recommended in all cases of cirrhosis. Hepatitis B and C may be treatable with antiviral medications. Autoimmune hepatitis may be treated with steroid medications. Ursodiol may be useful if the disease is due to blockage of the bile ducts. Other medications may be useful for complications such as abdominal or leg swelling, hepatic encephalopathy, and dilated esophageal veins. In severe cirrhosis, a liver transplant may be an option.

Cirrhosis affected about 2.8 million people and resulted in 1.3 million deaths in 2015. Of these deaths, alcohol caused 348,000, hepatitis C caused 326,000, and hepatitis B caused 371,000. In the United States, more men die of cirrhosis than women. The first known description of the condition is by Hippocrates in the 5th century BCE. The term cirrhosis was invented in 1819, from a Greek word for the yellowish color of a diseased liver.

Viral Hepatitis

Viral hepatitis is liver inflammation due to a viral infection. It may present in acute form as a recent infection with relatively rapid onset, or in chronic form.

The most common causes of viral hepatitis are the five unrelated hepatotropic viruses hepatitis A, B, C, D, and E. Other viruses can also cause liver inflammation, including cytomegalovirus, Epstein-Barr virus, and yellow fever. There also have been scores of recorded cases of viral hepatitis caused by herpes simplex virus.

The most common types of hepatitis can be prevented or treated. Hepatitis A and hepatitis B can be prevented by vaccination. Effective treatments for hepatitis C are available but costly.

In 2013, about 1.5 million people died from viral hepatitis, most commonly due to hepatitis B and C. East Asia is the region most affected.

### Combinations

The iPSC-derived Kupffer cell may be combined with any other cell type for use. Suitable cell types may include liver cell type such as a hepatocyte.

The hepatocyte may comprise a cell from a known hepatocyte cell line such as hepg2. It may comprise any other hepatocyte source as known in the art, for example heparg.

The hepatocyte may comprise a primary human hepatocyte (pHEP) or an iPSC-derived hepatocyte (iHep).

The cell type may share a genetic background with the iPSC-derived Kupffer cell. For example the iPSC-derived Kupffer cell and the cell type in the combination may be derived from the same do not or donor matched.

The iPSC-derived Kupffer cell and the cell type in the combination may be cultured together, for example in a co-culture.

### EXAMPLES

Common cell culture consumables and growth factors were obtained from Life Technologies (Carlsbad, Calif., USA) and R&D Systems (Minneapolis, Minn., USA) respectively, unless stated otherwise.

### Example 1. Materials and Methods: Cell Culture

iPSC-IMR90 (WiCell Research Institute, Madison, Wis.) was cultured on matrigel (BD Biosciences, San Jose, Calif., USA)—coated tissue culture plates in mTeSR™1 media (Stem Cell Technologies, Vancouver, BC, Canada) and maintained as described previously [24, 25].

Cryopreserved pKCs (Life Technologies) were maintained and cultured according to manufacturer's instructions with modifications. Briefly, the cells were thawed in a 37° C. water bath and resuspended in cold KC Thawing/Plating Medium comprising of Advanced DMEM, 5% FBS and supplement cocktail A (Life Technologies). Major components of the cocktail include: human recombinant insulin (4 μg/ml), glutamax (2 mM), HEPES (15 mM) and 100 U/mL penicillin and 100 mg/mL streptomycin. The cells were centrifuged at 150 g at 4° C. and the cell pellet was resuspended in KC Thawing/Plating Medium. Cells were counted and 12,000 cells were plated into each well of 96-well plates (Nunc, Naperville, Ill., USA) pre-coated with neutralized 1.5 mg/ml PureCol® Bovine Collagen solution, Type 1 (Advanced Biomatrix, San Diego, Calif., USA). After allowing for cell attachment for 24 hours, the media was changed to KC Maintenance media comprising of Advanced DMEM, 5% FBS and supplement cocktail B (Life Technologies) and the cells were either used directly for assays or co-cultured with hepatocytes. Major components of cocktail B include: human recombinant insulin (6.25 μg/ml), human transferrin (6.25 ng/ml), selenous acid (6.25 ng/ml), bovine serum albumin (1.25 mg/ml), linoleic acid (5.35 μg/ml), glutamax (2 mM), HEPES (15 mM) and 100 U/mL penicillin and 100 mg/mL streptomycin.

Cryopreserved pHeps were obtained from Life Technologies and BD Biosciences (Franklin Lakes, N.J., USA) and cultured as previously described [24]. Culture medium was changed daily. The media collected from the cultures was used as primary human hepatocyte conditioned media (PHCM). Three different lots of pHeps and pKCs were used for the experiments.

### Example 2. Materials and Methods: In Vitro Differentiation of iHeps, preMφ, iKCs and NL-Mφ

iPSCs were differentiated into hepatocytes as described previously [26]. At least three independent batches of differentiated iPSC-derived hepatocytes (iHeps) were used for all assays. Once differentiation was completed, cells were harvested using previously optimized protocol [25] for further experiments.

iPSC—derived macrophage precursors (preMφ), were generated using a protocol adopted from Wilgenburg et al. [26]. Briefly, iPSCs were harvested using TrypLE™, centrifuged and the cell pellet was resuspended in stem cell maintenance media mTeSR™1, supplemented with 50 ng/mL bone morphogenetic protein-4 (BMP-4, GenBank Accession Number Q53XC5), 50 ng/mL vascular endothelial growth factor (VEGF, GenBank Accession Number NP_001165097), 20 ng/mL stem cell factor (SCF, GenBank Accession Number P21583.1) and 10 μM ROCK Inhibitor (Calbiochem, Billerica, Mass., USA). The cells were seeded at a density of 12,000 cells/well into a round-bottom, low adherence, 96-well plate, which was centrifuged and incubated at 37° C. in a 5% CO₂ atmosphere for 4 days before harvesting the embryoid bodies (EBs). 75% media change was performed on the second day. At day 4, 12 EBs were harvested and transferred into each well of a 6-well plate and cultured in X-VIVO™15 media (Lonza, Basel, Switzerland), supplemented with 100 ng/mL macrophage colony stimulating factor (M-CSF, GenBank Accession Number P09603), 25 ng/mL Interleukin-3 (IL-3, GenBank Accession Number AAC08706), 2 mM glutamax, 100 U/mL penicillin and 100 mg/mL streptomycin and 0.055 mM β-mercaptoethanol (Sigma-Aldrich, Singapore). Two-thirds of the media was changed every five to seven days. preMφ were generated from the EBs in 3 to 4 weeks. Suspended preMφ were collected from the media weekly and used for further differentiation.

preMφ were differentiated into Mφ using X-VIVO™15 media, supplemented with 100 ng/mL M-CSF (GenBank Accession Number P09603), 2 mM glutamax and 100 U/mL penicillin and 100 mg/mL streptomycin [26]. The differentiation lasted took 5 to 7 days. These cells are referred to as non-liver macrophages (NL-Mφ) as no liver specific cues were provided during the differentiation process.

In order to identify the optimal culture conditions for generating iKCs, several different culture conditions were screened. These conditions included extracellular matrix substrate (with and without Collagen I), different basal media to be combined with PHCM (X-VIO 15, RPMI 1640 and Advanced DMEM), serum supplementation and combination of these factors. Following optimization, preMφ were subjected to a mixture of PHCM and Advanced DMEM (plus supplements) to generate iKCs. PHCM was collected from cultures of 3 different lots of pHeps. These 3 different lots of PHCM were used with three different batches of preMφ to generate three batches of iKCs. After 5 to 7 days of differentiation, the cells were ready for harvesting. These liver-specific-Mφ like cells derived from preMφ are referred to as iKCs.

### Example 3. Materials and Methods: Experimental Set Up of Mono-Culture and Co-Culture

For co-cultures, NL-Mφ, pKCs or iKCs were seeded at a density of 12,000 to 15,000 cells per well on Collagen I-coated 96-well plates. The cells were seeded in their respective media for 24 hours. Following cell attachment, medium was removed and iHeps or pHeps were seeded at a density of 2.5 times the density of the NL-Mφ/pKCs/iKCs in William's E media containing supplemental cocktail B. In some experiments, Advanced DMEM with supplement cocktail B (Media A) was used. The matching hepatocyte mono-culture controls were seeded in the same media and at the same density as the co-cultures.

### Example 4. Materials and Methods: Quantitative Real Time PCR (qPCR)

RNA was isolated using RNeasy Micro-kit (Qiagen, Hilden, Germany), RNA amount was determined using a NanoDrop™ ND-1000 Spectrophotometer (Life Technologies) and RNA was converted to cDNA using iScript cDNA synthesis kit (Bio-Rad Laboratories, Hercules, Calif., USA). 7000 Fast Real-Time PCR System (Applied Biosystems, Foster City, Calif., USA) was used for qPCR using primers commercially obtained from GeneCopoeia, Inc.

(Rockville, Md., USA). The expression levels of all marker genes were normalized to that of glyceraldehyde-3-phosphate dehydrogenase (GAPDH).

### Example 5. Materials and Methods: Immunofluorescence

Immunostaining was performed as described [24]. The following primary antibodies were used: rabbit anti-CD68 (Abcam, Cambridge, U.K), goat anti-albumin (Abcam), mouse anti-CD163 (AbD Serotec, Raleigh, N.C., USA)), mouse anti-CD32 (AbD Serotec), goat anti-CLEC4F (Santa Cruz Biotechnology Inc., Santa Cruz, Calif., USA) and mouse anti-CD11(Abcam). Alexa Fluor 488-conjugated anti-mouse (Life Technologies) and Alexa Fluor 555-conjugated anti-rabbit (Life Technologies) secondary antibodies were applied. Cell nuclei were stained with DAPI and imaging was performed with Olympus BX-DSU microscope (Olympus, Tokyo, Japan). Fluorescent intensity was measured using Image J software and number of fluorescently labeled cells and total number of cells (DAPI-stained) were used to calculate percentage of cells expressing specific markers. 10 representative images were used for the quantification.

### Example 6. Materials and Methods: Flow Cytometry

Cells for flow cytometry analysis were prepared using standard staining procedures. The same antibodies used for immunofluorescence were used for flow cytometry. Positive gates were defined by unstained samples and isotype control. Data was acquired by LSRFortessa (BD Bioscience) and analyzed by Flow Jo (Tree Star, Inc.).

### Example 7. Materials and Methods: Cytokine Production

Cells were treated with 100 ng/ml lipopolysaccharide (LPS) for 16 hours prior to collection of media. Interleukin-6 (IL-6) and Tumor necrosis factor alpha (TNFα) levels in the media were measured using human IL-6 and TNFα enzyme-linked immunosorbent assay (ELISA) kits (Abcam) according to manufacturer's instructions.

### Example 8. Materials and Methods: Phagocytosis

To measure phagocytosis, FluoSpheres carboxylate-modified microspheres, 1.0 μm, were added at a ratio of 2 particles per cell in respective serum-free cell culture media. Following a 30 minutes incubation at 37° C., cells were washed with PBS, to remove particles bound to the outside of the cell. Fixation was carried out using 3.7% formaldehyde and stained with rabbit anti-CD163 to quantify number of cells in each field of image. Co-localization of fluorescent beads with CD163 staining was used to quantify percentage of phagocytosing cells. Beads uptaken by each cell were counted and normalized to number of cells in each field of image to obtain average number of beads per cell. Uptake of particles was quantified using Zeiss LSM 510 Meta confocal microscope (Carl Zeiss, Jena, Germany) to ensure that only beads that were uptaken were counted and beads attached to the surface of the cell were excluded. Three independent batches of experiments were performed (n=3) and at least 10 images from each batch were analyzed for quantification of phagocytosis.

### Example 9. Materials and Methods: Cell Viability Assays

Cell viability of differentiated hepatocytes was measured 24 hours after treatment with drugs using Alamar Blue® cell viability assay (Life Technologies) according to manufacturer's instructions. The following drugs were used for the cell viability assays: Acetaminophen, Trovafloxacin and Levofloxacin (all drugs from Sigma). Stock solutions of drugs were prepared in DMSO and then diluted in media to obtain desired working concentrations. Controls were treated with solvent alone (in absence of test compounds) and considered as 100% viability value.

### Example 10. Materials and Methods: Microarray Analysis

RNA was isolated using the RNeasy Micro-kit (Qiagen), and sent to the microarray facility, Institute of Molecular and Cellular Biology, Agency for Science, Technology and Research, Singapore, where the quality of RNA samples were analyzed using Bioanalyzer 2100 (Agilent) and microarrays were performed using the GeneChip Human Gene 2.0 ST arrays (Affymetrix). Expression Console software (Affymetrix) was used to normalize data (probe set summarization, initial data quality examination, background correction and log 2-transformation). The normalized data was analyzed using Transcriptome Analysis Console. Previous published data for IMR90 [27], BMDMs [28], BMDM-Mφ [29], alveolar Mφ [30], microglia [31] was also included in the data analysis. Common genes were first identified and principal component analysis (PCA) and clustering analysis were then performed using R (Version 3.3.2). For hierarchical clustering, Euclidean distance measurement and complete agglomeration method were applied. All microarray data have been deposited at the National Center for Biotechnology Information Gene Expression Omnibus public database under accession number GSE99734.

### Example 11. Materials and Methods: Cytochrome P450 (CYP) Activity

At day 5 of culture, basal activity of three CYP enzymes, i.e., CYP1A2, CYP2B6, and CYP3A4, were determined as described previously [25]. Cells were incubated with medium containing CYP-specific substrates (CYP1A2: 200 μM phenacetin, CYP2B6: 200 μM bupropion, and CYP3A4: 5 μM midazolam). The substrates were obtained from Sigma. The drug metabolite products in the supernatant was analyzed by liquid chromatography—mass spectrometry (LC-MS Finnigan LCQ Deca XP Max, Agilent 1100 series) according to procedures described previously [32].

### Example 12. Materials and Methods: Bile Acid Accumulation

iHeps-iKCs co-culture and iHeps mono-culture were treated with 100 ng/ml LPS and 10 μM Chlorpromazine (CPZ) for 24 hours. Cells were incubated with 10 μM Fluorescein diacetate (FDA) and incubated for 30 minutes. The FDA-containing medium was removed and the cells were washed thrice with medium to remove any residual FDA. Images of bile acid accumulation were acquired using Olympus BX-DSU and quantified using Image J software and normalized to untreated control.

### Example 13. Materials and Methods: Statistical Analysis

3 independent batches of differentiation for iPSC-derived cells: iHeps, NL-Mφ and iKCs and 3 independent lots (donors) for pHeps and pKCs were used. Two-way paired Student's t test was used for pair-wise statistical comparisons and ANOVA was used for microarray data analysis (integrated in the Transcriptome Analysis Console). Results are expressed as mean±standard error of the mean (s.e.m) of 3 independent experiments.

### Example 14. Results: iPSCs Differentiation into iKCs Via preMφ

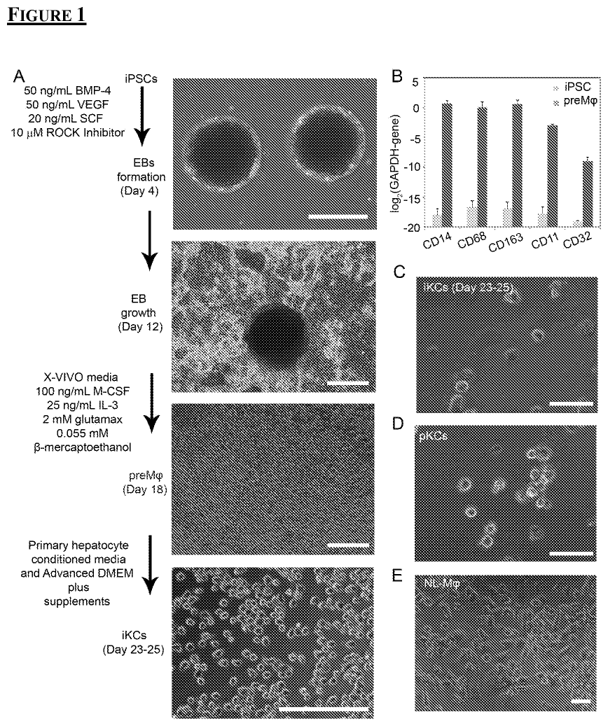

preMφ were differentiated from iPSC-IMR90 according to methods adopted from van Wilgenburg et al. [26]. Embryoid bodies (EBs) were formed and maintained in culture under serum-free and feeder-free conditions (FIG. 1A). preMφ production started at approximately 18 days and could be harvested weekly from the supernatant of the differentiation cultures. The EB formation, maintenance, adherence and generation of preMφ was consistent with previous reports [26]. preMφ expressed Mφ markers CD14, CD68, CD163, CD11 and CD32, which were expressed at very low levels (<0.1 fold) in iPSCs (FIG. 1B).

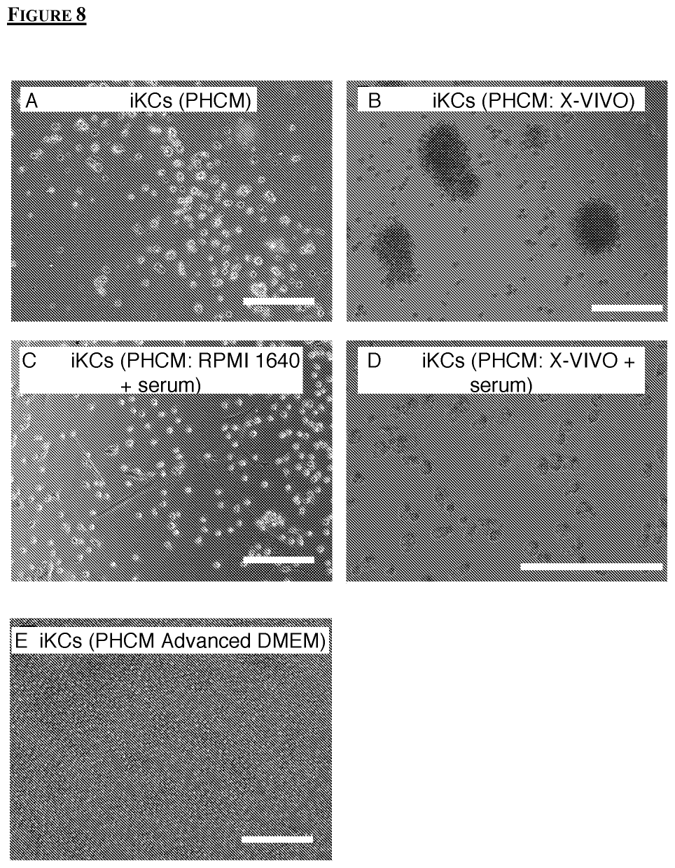

In order to differentiate preMφ to iKCs, we identified optimal culture conditions to be used in combination with PHCM. These conditions included optimal substrate, serum concentration and basal media. Addition of PHCM alone resulted in poor cell viability and attachment of iKCs, as shown by the unhealthy and disintegrating morphology (Supplementary FIG. 1A). Unhealthy cell morphology was not improved by a combination of PHCM with X-VIVO (which has been used in established protocols for Mφ differentiation [26]), with or without collagen (representative image shown in FIG. 8B). The cells either clustered together in large aggregates or appeared to be disintegrating. Similar results were obtained when RPMI 1640 was used instead of X-VIVO (data not shown). Addition of 10% serum to the media also didn't not greatly improve the morphology of the cells and surprisingly did not help in cell attachment (less than 50% of the cells were still attached at day 5 of differentiation; FIG. 8C, FIG. 8D). Interestingly, treatment of preMφ with a combination of PHCM and Advanced DMEM resulted in healthy cell morphology and attachment (FIG. 8E). Therefore, we used this combination to generate iKCs from preMφ (FIG. 1A). Media was changed every 3 days. The morphology of iKCs was comparable to pKCs, with similar diameter and phenotype (FIG. 1C, FIG. 1D) and different from NL-Mφ, which showed a more elongated shape and spread morphology (FIG. 1E). Consecutively, we performed microarray, gene expression analysis, immunostaining and functional assays to assess if iKCs were indeed similar to pKCs and different from NL-Mφ.

### Example 15. Results: iKCs Express Mφ and KC-Specific Markers at Levels Similar to pKCs: Gene Expression

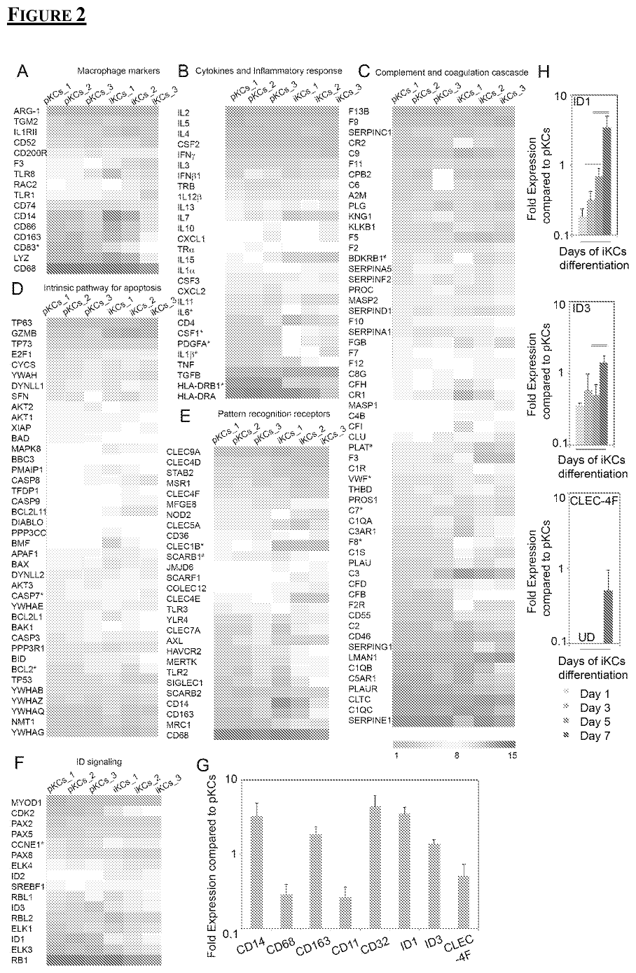

To assess similarity of iKCs and pKCs on a global gene expression level, we used microarray to analyze the expression of Mφ markers and pathways important for KCs functions: genes involved in complement and coagulation cascade, apoptosis, cytokine and inflammatory response, pattern recognition and Inhibitor of DNA binding proteins (ID) signaling. Gene expression in iKCs was compared to that of pKCs. iKCs expressed key Mφ markers such as CD163, CD200R and CD86 at levels similar to pKCs, with the exception of CD83, which was upregulated in pKCs by 4.4, fold (FIG. 2A). 91.5% of the genes involved in the key functional pathways had similar expression in pKCs and iKCs (differential expression based on at least 2-fold change and p-value<0.05). Only 15 out of 188 analyzed genes were differentially expressed in pKCs and iKCs (FIG. 2B to FIG. 2F, Table 1).

TABLE 1
Summary of genes involved in KC functional pathways
that are upregulated and downregulated in pKCs and
iKCs. Heatmaps showing expression of genes involved
in the pathways are presented in FIGS. 2A to 2F.

| | | # of genes | # of genes |
|---|---|---|---|
| | # of genes | upregulated | upregulated |
| Pathways | analysed | in pKCs | in iKCs |
| Macrophage markers | 16 | 1 | 0 |
| Cytokines and inflammatory | 28 | 5 | 0 |
| response | | | |
| Complement and coagulation | 59 | 3 | 2 |
| cascade | | | |
| Intrinsic pathway for apoptosis | 41 | 2 | 0 |
| Pattern recognition receptors | 28 | 1 | 1 |
| ID signalling | 16 | 0 | 1 |
| Total genes | 188 | 12 | 4 |

These 15 genes included 11 genes upregulated in pKCs: IL-6, colony-stimulating factor 1, platelet-derived growth factor subunit A, interleukin 113 and major histocompatibility complex, class II (HLA-DRB1) involved in cytokine and inflammatory response (4.7, 3.1, 2.9, 7.8 and 320 fold respectively; FIG. 2B), complement C7, coagulation factor 8 and von Willebrand factor, involved in complement and coagulation cascade (10.3, 8.0 and 9.0 fold respectively; FIG. 2C), caspase 7 and b-cell lymphoma 2 involved in apoptosis (2.3 and 6.2 fold respectively; FIG. 2D) and C-Type lectin domain family 1 member B involved in pattern recognition upregulated by 10 folds (FIG. 2E). Plasminogen activator and bradykinin receptor B1 involved in complement and coagulation cascade (FIG. 2C), scavenger receptor class B member 1 involved in pattern recognition (FIG. 2E) and cyclin E1 (ID signaling, FIG. 2F) were upregulated in iKCs by 2.4, 9.7, 3.73 and 3.3 folds respectively. There were no significant differences in expression level of the remaining 172 genes involved in these pathways between pKCs and iKCs. Overall, these results demonstrate the transcriptomic similarity between iKCs and pKCs.

We then confirmed expression of key Mφ and KC-specific genes by qPCR. F4/80 has been well-documented as a representative marker for mouse KCs but not for human cells [18]. CD14 in combination with a classification of CD32, CD68 and CD11 subpopulations of KCs have been used to define KCs in humans [33]. CD163 has been used as a marker for activated Mφ. Fold expression of iKCs compared to pKCs was 3.2±1.6 for CD14, 1.8±0.5 for CD163 and 4.3±1.8 for CD32 (FIG. 2G). CD68 and CD 11 expression in iKCs was approximately 30% of pKCs. In addition to these Mφ-specific markers, iKCs also expressed KC-specific markers: ID1 [34], ID3 [34] and C-Type Lectin Domain Family 4 Member F (CLEC-4F) [35, 36] at levels comparable to pKCs. Fold expression of iKCs compared to pKCs was 3.5±0.7 for ID1, 1.4±0.2 for ID3 and 0.5±0.2 for CLEC-4F (FIG. 2G).

To confirm that the expression of KC-specific markers were indeed due to progression of differentiation from preMφ to iKCs, we further examined the expression kinetics of KC-specific markers throughout the differentiation period by qPCR. ID was upregulated 1.8±0.8 fold between day 1 and day 3, 2.2±0.7 fold between day 3 and day 5 and 3.9±0.8 fold between day 5 and day 7 (FIG. 2H). Overall upregulation of ID1 during the differentiation period was 14±5 (day 7 vs day 1). ID3 was upregulated 3.7±0.4 folds at day 7 when compared to day 1 and 3.4±0.96 folds when compared to day 5 (FIG. 2H). CLEC-4F was not detected at days 1-5 but was detected at day 7 at levels similar to pKCs (FIG. 2H). Overall iKCs are mature in terms of Mφ and KC-specific marker expression which gradually increased during the seven-day differentiation period.

### Example 16. Results: iKCs Express Mφ and KC-Specific Markers at Levels Similar to pKCs: Protein Expression

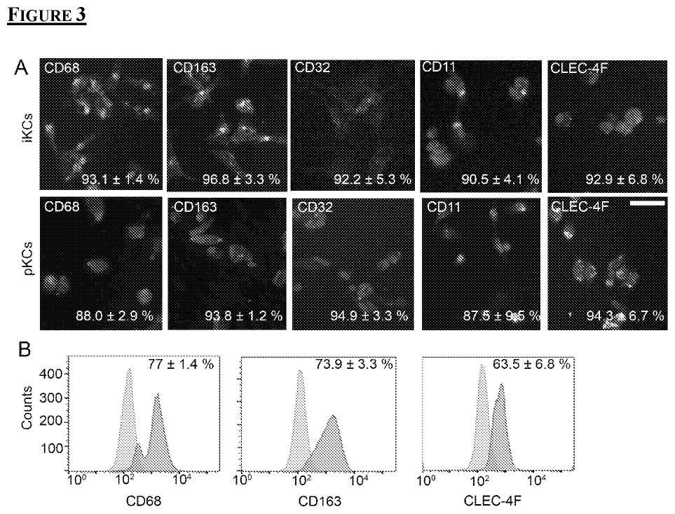

We analyzed similarity between iKCs and pKCs at a protein level by immunostaining (FIG. 3A). iKCs, similar to pKCs, expressed Mφ-specific markers and KC-specific marker CLEC-4F at protein level. Percentage of cells expressing Mφ-specific markers were quantified: CD68⁺: iKCs 93.1±1.4% and pKCs 88.0±2.9%, CD163⁺: iKCs 96.8±3.3% and pKCs 93.8±1.2%, CD11⁺: iKCs 90.5±4.1% and pKCs 87.5±9.5%, CD32⁺: iKCs 92.2±5.3% and pKCs 94.9±3.3%, CLEC-4F⁺: iKCs 92.9±6.8% and pKCs 94.3±6.7%. The immunofluorescence results were confirmed using flow cytometric analysis of key macrophage markers, CD68 and CD163 and KC-specific marker CLEC-4F in iKCs (FIG. 3B). The percentage of CD68⁺, CD163⁺ and CLEC-4F⁺ iKCs were 77±1.4%, 73.9±3.3% and 63.5±6.8%. Overall, iKCs and pKCs showed similar marker expression at a protein level.

### Example 17. Results: iKCs Express Liver Specific Macrophage Markers Absent in Non-Liver Macrophages

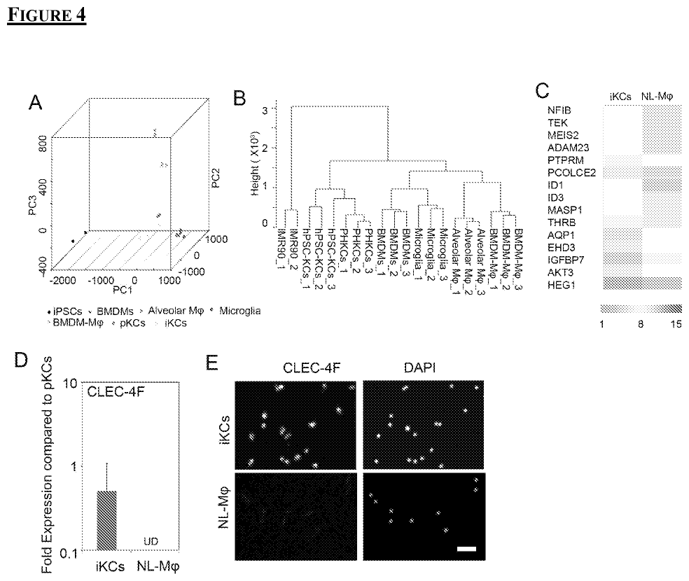

We used transcriptome microarray analysis to compare the molecular signatures of iKCs to BMDM-Mφ [29] and non-liver tissue resident Mφ: alveolar-Mφ [30] and microglia [31] from available public databases. iPSC-IMR90 [27] (stem cell source of iKCs), BMDMs [28], and pKCs (positive control) were included in the analysis. Based on Principal Component Analysis (PCA), iKCs and pKCs separated into a distinct group compared to iPSCs and BMDMs, as well as microglia, alveolar-Mφ and BMDM-Mφ (FIG. 4A). This was confirmed by hierarchical clustering of the samples, which showed that iKCs clustered with pKCs and not with iPSCs, BMDMs or non-liver macrophages (FIG. 4B). Overall, the global gene expression analysis suggested that the expression pattern of iKCs is similar to pKCs but different from other non-liver Mφ.

As a set, genes previously identified to be associated specifically with liver-specific Mφ (KCs) but not with other non-liver tissue-resident Mφ populations [34, 35] revealed that iKCs expressed 2 to 16 folds expression of these markers as compared to NL-Mφ generated in our lab (FIG. 4C). In this regard, NL-Mφ were included to demonstrate that a stem cell differentiation protocol without liver-specific cues (NL-Mφ) did not yield cells with functions same as iKCs. These results, combined with the PCA and clustering analysis showing differential molecular signatures in iKCs compared to alveolar Mφ or microglia (FIG. 4A, FIG. 4B), suggest that iKCs indeed express liver Mφ-specific markers as opposed to other non-liver-resident Mφ markers. In summary, the microarray analysis revealed a high degree of similarity between iKCs and pKCs (FIG. 4A, FIG. 4B and FIG. 2A to FIG. 2F) than between iKCs and NL-Mφ or Mφ in the brain or lungs, indicating liver-specificity of iKCs.

We confirmed the differences in marker expression between iKCs and NL-Mφ through gene and protein expression. We have reported the expression of three KC-specific markers: ID1 and ID3 and CLEC-4F in iKCs (FIG. 2G, FIG. 2H). Out of these markers, CLEC-4F is a key marker which is expressed differentially in KCs compared to other Mφ [35, 36]. Therefore, we analyzed if pKCs and NL-Mφ differed in CLEC-4F expression. CLEC-4F was expressed in iKCs, but not in NL-Mφ both at gene expression level shown by qPCR (FIG. 4D) and at protein level as shown by immunofluorescence (FIG. 4E). Gene expression of CLEC-4F in iKCs was 51% of pKCs on average, yet insignificant due to varying levels of CLEC-4F expression in different lots of pKCs examined. Together this shows the liver-specific marker expression of iKCs, which is similar to pKCs but different from NL-Mφ.

### Example 18. Results: Functions of iKCs are Similar to pKCs and Different from NL-Mφ

We determined if iKCs are similar to pKCs and different from NL-Mφ at a functional level. KCs exhibit a higher level of phagocytosis and lower level of cytokine production compared to NL-Mφ [37, 38]. We examined if the level of phagocytosis was different in iKCs and NL-Mφ. iKCs, pKCs and NL-Mφ were incubated with fluorescent beads for one hour and the number of phagocytosed beads was analyzed using a confocal fluorescence microscope.

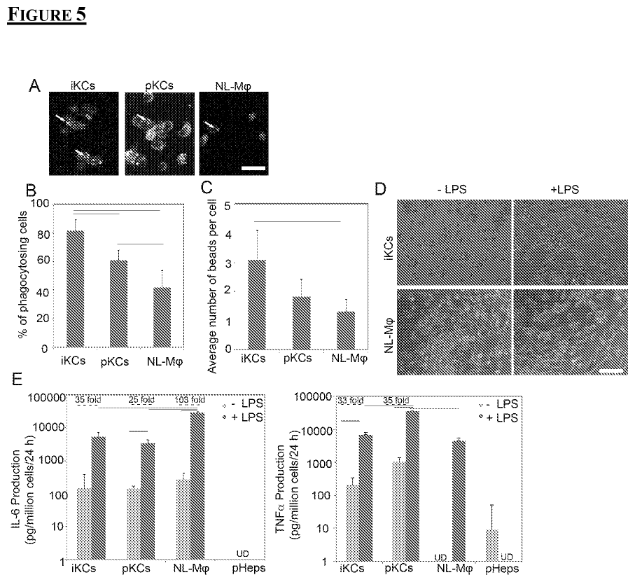

All three cell types: iKCs, pKCs and NL-Mφ engulfed the beads (FIG. 5A). A higher percentage of iKCs (82±8%) and pKCs (61±7%) phagocytosed the beads when compared to NL-Mφ (42±12%) (FIG. 5B). The average number of beads taken up by the cells was also higher in iKCs (3 beads per cell) when compared to NL-Mφ (1 bead per cell) (FIG. 5C). The average number of beads uptaken by pKCs (2 beads per cell) was higher than NL-Mφ, although this difference was not statistically significant. Overall, iKCs, similar to pKCs, were more active in performing phagocytosis compared to NL-Mφ.

Due to the anatomical connection between the liver and intestines, KCs are the first cells to be exposed to gut-derived toxins including LPS. LPS binding protein (LBP) facilitates LPS-LBP complex formation and interaction with CD14 receptors on KCs which eventually leads to signal transduction via Toll-like receptor 4 (TLR4) [39]. TLR4 signaling drives KCs to produce an array of pro- and anti-inflammatory cytokines and chemokines [39]; TNFα and IL-6 being the most well-studied cytokines [3, 9, 40]. To examine the responsiveness of iKCs to LPS activation and TNFα and IL-6 production in vitro, we stimulated iKCs with 100 ng/ml LPS for 16 hours. pKCs and NL-Mφ were treated similarly. Culture media was collected at the end of the incubation period and morphological changes and cytokine production were analyzed. LPS activation induced typical morphological changes from round, to flat and spread in iKCs (FIG. 5D, upper panel). This phonotypical change was similar to that of NL-Mφ (FIG. 5D, lower panel). LPS activation induced a 35-fold increase in IL-6 production in iKCs (FIG. 5E). Importantly, the fold induction in iKCs was in the same range as that of the pKCs (25 folds). No significant differences were observed between IL-6 levels in iKCs and pKCs in terms of basal level (no LPS treatment) and LPS-induced level (basal iKCs and pKCs—148 and 139 pg/million cells/24 h respectively; LPS-treated: iKCs and pKCs—5260 and 3420 pg/million cells/24 h respectively). IL-6 production in pHeps was below detectable levels. iKCs showed increase in TNFα production upon treatment with LPS (FIG. 5E). The fold increase in TNFα production in iKCs (33 folds) was similar to the fold increase in pKCs (35 folds). TNFα production in pHeps upon LPS activation was below detectable levels. In contrast to pKCs and iKCs, NL-Mφ showed a much higher level of LPS-induced IL-6 and TNFα production (IL-6: 103 folds, TNFα: >1000 folds; exact fold induction values were not reported since levels without LPS treatment could not be detected, FIG. 5E). In summary, iKCs showed LPS-induced increase in cytokine production at levels similar to that of pKCs and these levels were much lower compared to the NL-Mφ, confirming that iKCs indeed demonstrate KC-like functionality.

### Example 19. Results: iKCs, Similar to pKCs, can be Co-Cultured with Hepatocytes for a Functional In Vitro Liver Model

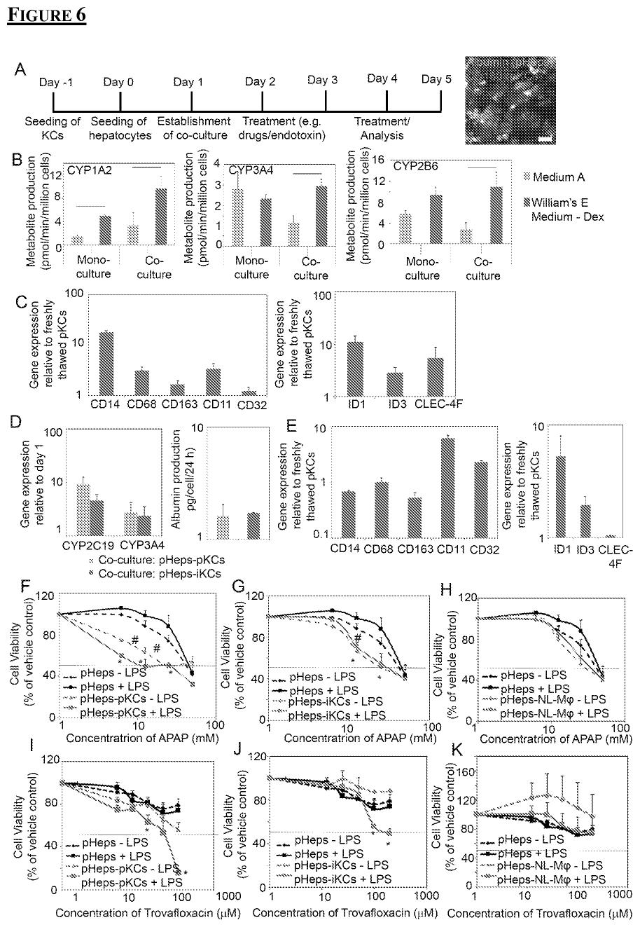

We investigated if iKCs could be co-cultured with hepatocytes without compromising the functionality of either cell type. Co-culture of pHeps and iKCs/pKCs was set up as shown in FIG. 6A. 24 hours after completion of cell seeding is considered as day 1 of co-culture, where optimal co-culture media was added and cells maintained for subsequent assays. When the model was used for applications such as drug testing, treatment would be initiated at day 2, and assays carried out at days 3-5, depending on the duration of the treatment (typically carried out for 24-72 hours).

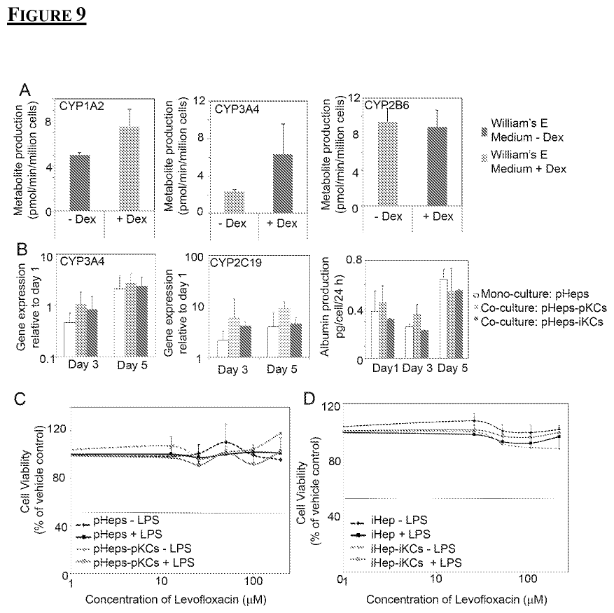

It is critical that functions of both hepatocytes and KCs are maintained in co-culture. Since hepatocytes and KCs have different media requirements, we first optimized media conditions for the co-culture. Dexamethasone (Dex) is important for hepatocyte viability and metabolic activity [29] but detrimental to KC functions, especially cytokine production [9]. The typical basal medium used for hepatocytes is William's E Medium whereas the manufacturer's recommended basal medium for KCs is Advanced DMEM. We tested the recommended Advanced DMEM based medium referred to as Media A, William's E Medium with and without Dex in mono-culture and co-culture. All other media components were the same. In both mono-culture of pHeps and co-culture of pHeps-pKCs, William's E Medium without Dex was superior to Media A in terms of CYP1A2, CYP3A4 and CYP2B6 basal activity, which are key markers of hepatocyte function (FIG. 6B). In co-culture, metabolite production upon treatment with CYP-specific substrates in Media A vs William's E Medium without Dex was 3.3±1.05 vs 9.7±2.1 (CYP1A2), 1.1±0.3 vs 2.9±0.3 (CYP3A4) and 2.8±1.2 vs 10.9±2.8 (CYP2B6) μmol/min/million cells. Although CYP1A2 and CYP3A4 activity was slightly higher in media containing Dex, the difference in metabolite production in media with and without Dex was not statistically significant (FIG. 9A), suggesting that removal of Dex from the media did not significantly compromise hepatocyte function. When pKCs were cultured in William's E Medium without Dex, Mφ marker and KC-specific marker expression were maintained or improved at day 5 when compared to freshly thawed pKCs (FIG. 6C). Mφ marker expression of CD14, CD68, CD163, CD11 and CD32 was 18.1±0.3, 3.1±1.7, 1.7±3.3, 3.3±0.9 and 1.2±0.2 folds compared to fresh pKCs respectively. Expression of KC-specific markers ID1, ID3 and CLEC-4F was 11.1±3.1, 2.8±0.7 and 5.5±3.2 folds compared to fresh pKCs respectively. These results suggest that William's E Medium without Dex is the optimal media for maintaining the function of both cell types, and used in subsequent experiments.

In order to determine if iKCs could be co-cultured with pHeps, we analyzed pHeps functions when co-cultured with iKCs and compared them to pHeps-pKCs co-culture at day 5 (FIG. 6D). Gene expression of CYP3A4 and CYP2C19 was normalized to expression levels at day 1. There was 2.4-fold upregulation of CYP3A4 and 4.7-fold upregulation of CYP2C19 at day 5 in pHeps-iKCs co-culture supporting that CYP function did not decline and even improved between days 1 and 5. The fold upregulation was similar to pHeps-pKCs co-culture (2.8 folds for CYP3A4 and 9.5 folds for CYP2C19). Albumin production was 1.6±0.4 and 1.7±0.01 pg/cell/24 h at day 5 in pHeps-pKCs and pHeps-iKCs co-culture respectively. Upon closer examination, no significant decline in CYP gene expression and albumin function was observed between days 1, 3 and 5 in pHeps-pKCs, pHeps-iKCs co-cultures and pHeps mono-culture (FIG. 9B). Mφ marker expression of CD14, CD68, CD163, CD11 and CD32 in iKCs was 0.7-6.1 folds and KC-specific marker expression was 5.3±2.7 (ID1), 1.9±0.4 (ID3) and 1.1±0.1 folds (CLEC-4F) compared to fresh pKCs (FIG. 6E). These results confirm that 1) including KCs to pHeps culture is not detrimental and can further improve functional performance of both hepatocytes and KCs; and 2) iKCs can be alternative cell source for pKCs in co-culture with hepatocytes.

### Example 20. Results: Toxicity Response in iKCs is Similar to iKCs and Different from NL-Mφ when Used in an Inflammatory Co-Culture Model

We analyzed if iKCs could be used in a liver model to detect inflammation-associated hepatotoxicity. pKCs, iKCs and NL-Mφ were co-cultured with pHeps and compared to pHeps mono-culture. The mono-culture and co-cultures were treated with endotoxin (LPS, to simulate inflammation) and paradigm hepatotoxicant Acetaminophen (APAP) for 24 hours; and cell viability was measured (FIG. 6F to FIG. 6H). Cell viability was quantified as the percentage of viable cells compared to vehicle control (DMSO). Higher cell death was observed in co-culture of pHeps-pKCs (FIG. 6F) and pHeps-iKCs (FIG. 6G) when compared to mono-culture controls, represented by a typical shift in the toxicity curve to the left. The IC₅₀ for pHeps-pKCs and pHeps-iKCs co-cultures was 12.5 mM and 25 mM respectively while that of mono-culture was 45 mM. No difference in cell death was observed between pHeps-NL-Mφ co-culture and pHeps mono-culture (FIG. 6H). These results suggest that pHeps-KCs co-culture represent a more sensitive model for hepatotoxicity testing compared to mono-culture. iKCs generated in our study can recapitulate the response shown by pKCs when co-cultured with pHeps. NL-Mφ cannot mimic this response in co-cultures. A similar trend was observed when cells were treated with Trovafloxacin (FIG. 6I to FIG. 6K). Higher cell death was observed in co-cultures of pHeps-pKCs (FIG. 6I) and pHeps-iKCs (FIG. 6J) when compared to mono-culture controls, which was more evident in concentrations higher than 50 μM. When cells were treated with 100 μM of Trovafloxacin, cell viability in pHeps-pKCs and pHeps-iKCs co-cultures was 53.8 and 55.8% respectively, while 73.8% of the hepatocytes were viable in the mono-culture. No difference in cell viability was observed between mono-culture and pHeps-NL-Mφ co-culture (FIG. 6K).

Overall iKCs, similar to pKCs, co-cultured with hepatocytes show differential toxicity responses to paradigm hepatotoxicants compared to hepatocyte mono-culture. This difference is not observed in hepatocytes co-cultured with NL-Mφ. Therefore, iKCs is a suitable alternative to pKCs as a cell source for hepatotoxicity testing under inflammatory conditions.

### Example 21. Results: iKCs can be Co-Cultured with iHeps for a Donor-Matched Inflammatory Model for Hepatotoxicity Testing

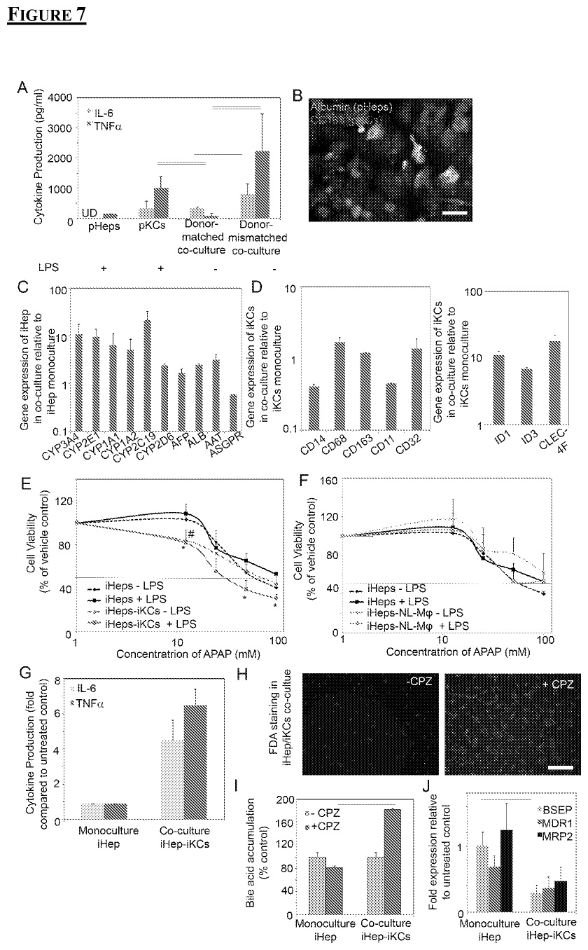

Donor-matched cell source of hepatocytes and KCs is important for avoiding background response elicited by KCs when cultured with donor-mismatched hepatocytes [9]. Such background reactions involve immune cells being activated even in the absence of a specific stimulant such as endotoxin and can affect the model specificity. Although such mismatch-induced response classically involves recognition by lymphocytes or Natural Killer cells, we investigated if donor-matched cell source is important for hepatocytes-KCs co-culture. Our data showed that cytokine production (IL-6 and TNFα) was elevated in donor-mismatched co-culture of pHeps-pKCs (primary human hepatocytes and KCs from different donors) even without endotoxin treatment (FIG. 7A). Donor-matched co-cultures (hepatocytes and KCs derived from the same iPSC source) did not show such cytokine elevation (FIG. 7A). pHeps mono-culture did not show any endotoxin stimulation even after LPS addition, suggesting that elevated cytokine levels in donor-mismatched co-culture is due to KC-response. The elevation in cytokine production in donor-mismatched culture is supported by previous report [41]. Since cytokine production by KCs can impact hepatocyte function [42], it is important to avoid background activation in donor-mismatched co-cultures in absence of any stimulation. Hence, donor-matched co-culture is important for hepatotoxicity and liver disease modelling.

To generate donor-matched stem-cell derived hepatocytes and KCs co-culture model, we co-cultured iPSC (IMR90)-derived hepatocytes (iHeps) with iKCs derived from the same iPSC-IMR90 line (FIG. 7B). With the exception of asialoglycoprotein receptor (ASGPR), gene expression of key hepatic markers showed an upregulation of 2.4-10.8 folds in iHeps-iKCs co-culture when compared to iHeps mono-culture, suggesting that co-culture with iKCs help to improve iHeps functions (FIG. 7C). With the exception of CD14 and CD11, gene expression of Mφ markers and KC-specific markers was respectively 1.2-1.7 folds and 6.9-18.1 folds higher in iKCs when co-cultured with iHeps compared to iKCs mono-culture (FIG. 7D). Thus, iHeps and iKCs could be co-cultured in a donor-matched model where the functions of individual cell types could be maintained or improved.

The donor-matched co-culture model (iHeps-iKCs) was further treated with APAP and cell viability measured. Without LPS activation, there was no significant difference between the mono-culture and donor-matched co-culture (FIG. 7E), suggesting minimal background crosstalk. This is in contrast to donor-mismatched co-culture of pHeps-pKCs and pHeps-iKCs (FIG. 6F and FIG. 7G) where a response was seen in co-cultures even without LPS addition. A change in cell viability was observed in donor-matched iHeps-iKCs only after LPS addition (IC₅₀: 27 mM) compared to mono-culture (IC₅₀: 54 mM) (FIG. 7E). This suggests that the donor-matched model is more specific and minimizes background response observed in donor-mismatched co-cultures. No difference in cell viability or IC₅₀ was observed when NL-Mφ were used instead of iKCs for co-culture with IMR90-derived hepatocytes (FIG. 7F), suggesting that the model requires iKCs and not just any Mφ-like cells. We also tested a negative compound, Levofloxacin under similar conditions to that of the positive compound APAP. Both primary and stem-cell derived cultures showed greater than 90% cell viability in mono-culture and co-culture of hepatocytes and iKCs (FIG. 9C, FIG. 9D), suggesting that differences in dose responses were specific to hepatotoxicants.

Overall these results show that 1) donor-matched co-culture model was successfully established using iKCs and iHeps. Performance of both cell types were maintained or improved in co-culture; 2) donor-matched co-culture model showed less background response compared to donor-mismatched culture; and 3) the dose responses were specific to iKCs (NL-Mφ did not show similar response) and to paradigm positive hepatotoxicants (no toxicity was observed with non-toxic compound).

### Example 22. Results: iHeps-iKCs Co-Culture can be Used to Model Cholestasis

KCs have been shown to be involved in the pathogenesis of cholestatic [43]. In vivo cholestatic model of bile duct ligation has been used to demonstrate changes in functional activity, such as elevated cytokine secretion in KCs [43]. Cytokine production in KCs can in turn affect transporter expression in hepatocytes [42]. In vitro co-culture of pHeps and nonparenchymal cells has shown bile acid accumulation and downregulation of bile transporters [4]. To determine whether the iHeps-iKCs co-culture model can be used to study specific liver-disease such as cholestasis, paradigm cholestasis-inducing compound, CPZ was added to activate iHeps-iKCs co-culture. IL-6 and TNFα production was increased by 4.5 and 6.5 fold respectively compared to untreated control (FIG. 7G). Significant bile acid accumulation was observed, indicating compromised bile acid transport, a key event in cholestasis (FIG. 7H, FIG. 7I). There was a concurrent decrease in mRNA expression of bile salt export pump (BSEP): 0.3±0.1 folds, multidrug resistance (MDR1): 0.4±0.2 folds and multidrug resistance-associated protein 1 (MRP1): 0.5±0.2 folds (FIG. 7J), suggestive of a potential mechanism for the accumulation [44, 45]. Bile acid accumulation, cytokine production and reduction in transporter gene expression were observed only in the co-culture but not in mono-culture (FIG. 7G, FIG. 7I, FIG. 7J). These results suggest that the iHeps-iKCs co-culture model can reproduce cholestatic pathologies in vitro and might be suitable for study underlying disease mechanisms and contribute to anti-cholestatic drug screening.

### Example 23. Discussion

This is the first report of a method for generating mature, functional human KCs from stem cells (iKCs). iKCs would be a crucial tool for the development of an inflammatory in vitro model that can mimic basal and inflammatory states of the liver. Our results showed that iKCs, similar to pKCs, exhibited Mφ-like and KC-specific markers and functions which were different from NL-Mφ. Co-culture model with hepatocytes established using these iKCs showed differential response to paradigm hepatotoxicants and paradigm cholestatic agent when compared to mono-culture.

The origin of KCs has been disputed. Based on the mononuclear phagocyte system [17], a foundational and prevailing dogma was that tissue-resident Mφ are derived from BMDMs. In contrast, several recent studies have reported that many tissue-resident Mφ populations, including KCs are derived from embryonic precursors during development and maintain themselves by self-renewal [19-21]. Recent studies have demonstrated common ontogeny between stem cell—derived Mφ and MYB-independent tissue resident Mφ [23]. Based on these evidences, tissue-specific Mφ (in this case, liver-specific Mφ, i.e. KCs) generated from stem cell-derived preMφ, as in our study, mimics the natural steps in development.

One important advantage of this protocol of iKCs differentiation is that, upon pre Mφ, iKCs can be generated from the same culture on a weekly basis. We and previous report [26] have shown these cultures can be maintained for months, hence iKCs can also be generated from for long periods of time instead of restarting the differentiation process from the initial stem cell source for every batch. This allows time and cost saving equivalent to three weeks per batch of KC differentiation since preMφ production from iPSCs takes three weeks. This allows a high yield of iKCs per batch of stem cell culture. For example, if 0.6 million iPSCs are seeded in 6 wells (12 EBs; 1.2×10⁴ iPSCs per EB=0.1 million iPSCs per well), 1.2 million preMφ/1 million iKCs can be generated per week (approximately 80% differentiation efficiency) from 6 wells (0.2 million preMφ per well). Following 8 consecutive weeks of continuous monocytopoiesis, 9.6 million preMφ/8 million iKCs can be cumulatively generated from the same culture. The rate is consistent with previous report where preMφ were generated continuously in culture [26]. If preMφ could not be continuously generated, 0.6 million iPSCs seeded in the example above would lead to 1.2 million preMφ/1 million iKCs and a fresh culture would have to be set up to restart the differentiation process.

We compared the marker expression and functional activity of iKCs to that reported in the literature. CD14, CD11, CD32, CD68 and CD163 have been used as markers for human KCs [33]. Our gene expression analysis showed that iKCs expressed CD14, CD32 and CD163 at levels similar to that of pKCs. CD68 and CD11 expression was lower than pKCs. It has been reported that CD14⁺ KCs in the human liver can be classified into CD32⁺, CD68⁺ and CD11⁺ subsets and that CD11⁻CD32+ cells might represent resident liver KCs [33]. This might explain the lower levels of CD11 expression in our iKCs cultures. TNFα production upon LPS stimulation has been reported to be 4000 pg/ml in KCs [46]; in our study, iKCs produced 10,000 μg/ml of TNFα. The fold induction of TNFα (calculated by comparing cytokine levels before and after LPS stimulation) has been reported to be 5 folds [47] and 10 folds [48]. In our study, this fold induction was 35 folds and 33 folds for pKCs and iKCs respectively. IL-6 production upon LPS stimulation has been reported to be 800 pg/ml [46] and fold induction has been reported to be 34 folds [47] and 5 folds [48]. In our study, IL-6 production upon LPS stimulation was 2018 pg/ml and 5200 pg/ml and fold induction was 25 folds and 35 folds for pKCs and iKCs respectively. These comparisons in cytokine production indicate that iKCs produce cytokine at levels similar to reported values and pKCs. Reports on functional differences between KCs and NL-Mφ have suggested that KCs show a higher level of phagocytosis and lower level of cytokine production compared to other Mφ. [37, 38] iKCs developed in this study, similar to pKCs, produced lower levels of IL-6 and TNFα upon LPS stimulation compared to NL-Mφ (IL-6: iKCs—35 folds, pKCs—25 folds and NL-Mφ—103 folds; TNFα: iKCs—33 folds, pKCs—35 folds and NL-Mφ>1000 folds; FIG. 4F) and a higher level of phagocytosis.

Previous reports have shown the suppression of CYP expression and activity in hepatocytes when co-cultured with KCs and cytokines [49, 50]. Co-cultures of hepatocytes and KCs using animal cells [9] have shown that administration of paradigm hepatotoxicants in the presence of endotoxin stimulation increases the sensitivity of the model, i.e. hepatotoxicity is detected at lower concentrations, depicted by a typical left shift of the dose response curve.

This is not surprising since hepatotoxic drugs can activate KCs [51], which in turn can impact the function of hepatocytes [52]. However, reports of such studies in a human liver model are limited. Brief reports from companies such as Hepregen and Life Technologies have shown differences in toxicity responses between human hepatocytes/KCs co-cultures and hepatocyte mono-culture [47, 53]. Co-cultures of hepatocytes and iKCs developed in our study showed a left shift of the dose response curve when cultures were treated with APAP and Trovafloxacin. Interestingly, in co-culture of pHeps and pKCs, this shift was observed even when LPS was not added to the culture. One possible explanation is that co-cultures of cells from different donors (mismatched donors: pHeps and pKCs) might activate the KCs even without LPS treatment [9]. This is further supported by our results from the donor-matched co-cultures (hepatocytes and KCs both derived from iPSC-IMR90; FIG. 7) where a left shift was only observed when LPS was added to the system. Since donor-matched primary cells are limited in availability, using iKCs would provide the additional advantage of obtaining both hepatocytes and KCs from the same stem cell source. Such donor-matched cells could be useful for potential future application in personalized medicine.

iPSCs have been useful as a renewable alternative cell source for primary cells, and have allowed studies of their biology and applications such as cell therapy and drug testing. The major bottleneck in using iPSCs-derived cells is that they typically retain an immature phenotype. While previous work from various laboratories has demonstrated the ability to generate different cell lineages from iPSCs, obtaining mature adult-like cells remains a major challenge in the field. iPSC-derived cells, including beta cells [15], dendritic cells [14], lung cells [12], cardiomyocytes [16] and hepatocytes [13] show an immature or fetal phenotype. These immature cells might be useful for studying immature human tissues, but their use in applications which require mature adult-like cells remain limited. iKCs generated in our study are not only functional but also mature, as depicted by their similarity to commercially obtained adult pKCs. This allows the usage of these cells to studies that requite a mature phenotype.

### Example 24. Conclusions

We have demonstrated that iKCs generated in this study are functionally competent and similar to pKCs. iKCs represent a novel renewable cell source for human KCs and allow the use of such cells for human in vitro inflammatory liver models for hepatotoxicity testing and study of liver disease such as cholestasis. The application of iKCs could be further extended to develop models for other inflammation-associated liver diseases such as liver fibrosis and hepatocellular carcinoma and for personalized medicine

### REFERENCES

[1] M. Bilzer, F. Roggel, A. L. Gerbes, Role of Kupffer cells in host defense and liver disease, Liver Int. 26 (10) (2006) 1175-1186.
[2] G. Kolios, V. Valatas, E. Kouroumalis, Role of Kupffer cells in the pathogenesis of liver disease, World J. Gastroenterol. 12 (46) (2006) 7413-7420.
[3] T. R. Billiar, R. D. Curran, Kupffer cell and hepatocyte interactions: a brief overview, JPEN J. Parenter Enteral. Nutr. 14(5 suppl) (1990) 175S-180S.
[4] C. C. Bell, D. F. Hendriks, S. M. Moro, E. Ellis, J. Walsh, A. Renblom, L. F. Puigvert, A. C. Dankers, F. Jacobs, J. Snoeys, Characterization of primary human hepatocyte spheroids as a model system for drug-induced liver injury, liver function and disease, Sci. Rep. 6 (2016) 25187.
[5] R. Kostadinova, F. Boess, D. Applegate, L. Suter, T. Weiser, T. Singer, B. Naughton, A. Roth, A long-term three dimensional liver co-culture system for improved prediction of clinically relevant drug-induced hepatotoxicity, Toxicol. Appl. Pharmacol. 268 (1) (2013) 1-16.
[6] R. Evers, S. Dallas, L. J. Dickmann, O. A. Fahmi, J. R. Kenny, E. Kraynov, T. Nguyen, A. H. Patel, J. G. Slatter, L. Zhang, Critical review of preclinical approaches to investigate cytochrome P450—mediated therapeutic protein drug-drug interactions and recommendations for best practices: a white paper, Drug Metab. Dispos. 41 (9) (2013) 1598-1609.
[7] Y. S. Zinchenko, L. W. Schrum, M. Clemens, R. N. Coger, Hepatocyte and kupffer cells co-cultured on micropatterned surfaces to optimize hepatocyte function, Tissue Eng. 12 (4) (2006) 751-761.
[8] F. F. Tukov, J. F. Maddox, D. E. Amacher, W. F. Bobrowski, R. A. Roth, P. E. Ganey, Modeling inflammation—drug interactions in vitro: a rat Kupffer cell-hepatocyte coculture system, Toxicol. In Vitro 20(8) (2006) 1488-1499.
[9] K. A. Rose, N. S. Holman, A. M. Green, M. E. Andersen, E. L. LeCluyse, Co-culture of hepatocytes and Kupffer cells as an in vitro model of inflammation and drug-induced hepatotoxicity, J. Pharm. Sci. 105 (2) (2016) 950-964.
[10] R. Landmann-Suter, Generation and Use of a Mouse Kupffer Cell Line, ALTEX, 24 (2007) 42-45.
[11] G. Heuff, A. A. D. Van Loosdrecht, M. G. Betjes, R. H. Beelen, S. Meijer, Isolation and purification of large quantities of fresh human Kupffer cells, which are cytotoxic against colon carcinoma, Hepatology 21 (3) (1995) 740-745.
[12] M. Aurora, J. R. Spence, hPSC-derived lung and intestinal organoids as models of human fetal tissue, Dev. Biol. 420 (2) (2016) 230-238.
[13] M. Baxter, S. Withey, S. Harrison, C.-P. Segeritz, F. Zhang, R. Atkinson-Dell, C. Rowe, D. T. Gerrard, R. Sison-Young, R. Jenkins, Phenotypic and functional analyses show stem cell-derived hepatocyte-like cells better mimic fetal rather than adult hepatocytes, J. Hepatol. 62 (3) (2015) 581-589.
[14] E. Takacs, P. Boto, E. Simo, T. I. Csuth, B. M. Toth, H. Raveh-Amit, A. Pap, E. G. Kovacs, J. Kobolak, S. Benkö, Immunogenic Dendritic Cell Generation from Pluripotent Stem Cells by Ectopic Expression of Runx3, J. Immunol. 198 (1) (2017) 239-248.
[15] F. W. Pagliuca, D. A. Melton, How to make a functional β-cell, Development 140 (12) (2013) 2472-2483.
[16] C. Robertson, D. D. Tran, S. C. George, Concise review: Maturation phases of human pluripotent stem cell—derived cardiomyocytes, Stem Cells 31 (5) (2013) 829-837.
[17] R. van Furth, Z. A. Cohn, The origin and kinetics of mononuclear phagocytes, J. Exp. Med. 128 (3) (1968) 415.
[18] R.-i. Takezawa, Y. Watanabe, T. Akaike, Direct evidence of macrophage differentiation from bone marrow cells in the liver: a possible origin of Kupffer cells, J. Biochem. 118 (6) (1995) 1175-1183.
[19] E. G. Perdiguero, K. Klapproth, C. Schulz, K. Busch, E. Azzoni, L. Crozet, H. Garner, C. Trouillet, M. F. de Bruijn, F. Geissmann, Tissue-resident macrophages originate from yolk-sac-derived erythro-myeloid progenitors, Nature 518 (7540) (2015) 547-551.
[20] C. Schulz, E. G. Perdiguero, L. Chorro, H. Szabo-Rogers, N. Cagnard, K. Kierdorf, M. Prinz, B. Wu, S. E. W. Jacobsen, J. W. Pollard, A lineage of myeloid cells independent of Myb and hematopoietic stem cells, Science 336 (6077) (2012) 86-90.
[21] F. Ginhoux, S. Jung, Monocytes and macrophages: developmental pathways and tissue homeostasis, Nat. Rev. Immunol. 14 (6) (2014) 392-404.
[22] G. Hoeffel, J. Chen, Y. Lavin, D. Low, F. F. Almeida, P. See, A. E. Beaudin, J. Lum, I. Low, E. C. Forsberg, C-Myb+ erythro-myeloid progenitor-derived fetal monocytes give rise to adult tissue-resident macrophages, Immunity 42 (4) (2015) 665-678.
[23] J. Buchrieser, W. James, M. D. Moore, Human induced pluripotent stem cell-derived macrophages share ontogeny with MYB-independent tissue-resident macrophages, Stem Cell Reports 8 (2) (2017) 334-345.
[24] F. Tasnim, D. Phan, Y.-C. Toh, H. Yu, Cost-effective differentiation of hepatocyte-like cells from human pluripotent stem cells using small molecules, Biomaterials 70 (2015) 115-125.
[25] F. Tasnim, Y.-C. Toh, Y. Qu, H. Li, D. Phan, B. C. Narmada, A. Ananthanarayanan, N. Mittal, R. Q. Meng, H. Yu, Functionally enhanced human stem cell derived hepatocytes in galactosylated cellulosic sponges for hepatotoxicity testing, Mol. Pharm. 13 (6) (2016) 1947-1957.
[26] B. van Wilgenburg, C. Browne, J. Vowles, S. A. Cowley, Efficient, long term production of monocyte-derived macrophages from human pluripotent stem cells under partly-defined and fully-defined conditions, PLoS One 8 (8) (2013) e71098.
[27] L. Zimmerlin, T. S. Park, J. S. Huo, K. Verma, S. R. Pather, C. C. Talbot, J. Agarwal, D. Steppan, Y. W. Zhang, M. Considine, Tankyrase inhibition promotes a stable human naïve pluripotent state with improved functionality, Development 143 (23) (2016) 4368-4380.
[28] A. R. Abbas, D. Baldwin, Y. Ma, W. Ouyang, A. Gurney, F. Martin, S. Fong, M. van Lookeren Campagne, P. Godowski, P. Williams, Immune response in silico (IRIS): immune-specific genes identified from a compendium of microarray expression data, Genes Immun. 6 (4) (2005) 319-331.
[29] M. Beyer, M. R. Mallmann, J. Xue, A. Staratschek-Jox, D. Vorholt, W. Krebs, D. Sommer, J. Sander, C. Mertens, A. Nino-Castro, High-resolution transcriptome of human macrophages, PLoS One 7 (9) (2012) e45466.
[30] S. Worgall, A. Heguy, K. Luettich, T. P. O'Connor, B.-G. Harvey, L. E. Quadri, R. G. Crystal, Similarity of gene expression patterns in human alveolar macrophages in response to Pseudomonas aeruginosa and Burkholderia cepacia, Infect. Immun. 73 (8) (2005) 5262-5268.
[31] R. Rock, S. Hu, A. Deshpande, S. Munir, B. May, C. Baker, P. Peterson, V. Kapur, Transcriptional response of human microglial cells to interferon-γ, Genes Immun. 6 (8) (2005) 712-719.
[32] B. Nugraha, X. Hong, X. Mo, L. Tan, W. Zhang, P.-M. Chan, C. H. Kang, Y. Wang, L. T. Beng, W. Sun, Galactosylated cellulosic sponge for multi-well drug safety testing, Biomaterials 32 (29) (2011) 6982-6994.
[33] M. Ikarashi, H. Nakashima, M. Kinoshita, A. Sato, M. Nakashima, H. Miyazaki, K. Nishiyama, J. Yamamoto, S. Seki, Distinct development and functions of resident and recruited liver Kupffer cells/macrophages, J. Leukoc. Biol. 94 (6) (2013) 1325-1336.
[34] E. Mass, I. Ballesteros, M. Farlik, F. Halbritter, P. Gunther, L. Crozet, C. E. Jacome-Galarza, K. Handler, J. Klughammer, Y. Kobayashi, Specification of tissue-resident macrophages during organogenesis, Science, 353 (6304) (2016) aaf4238.
[35] C. L. Scott, F. Zheng, P. De Baetselier, L. Martens, Y. Saeys, S. De Prijck, S. Lippens, C. Abels, S. Schoonooghe, G. Raes, Bone marrow-derived monocytes give rise to self-renewing and fully differentiated Kupffer cells, Nat. Commun. 7 (2016) 10321.
[36] C.-Y. Yang, J.-B. Chen, T.-F. Tsai, Y.-C. Tsai, C.-Y. Tsai, P.-H. Liang, T.-L. Hsu, C.-Y. Wu, M. G. Netea, C.-H. Wong, CLEC4F is an inducible C-type lectin in F4/80-positive cells and is involved in alpha-galactosylceramide presentation in liver, PLoS One 8 (6) (2013) e65070.
[37] D. L. Laskin, B. Weinberger, J. D. Laskin, Functional heterogeneity in liver and lung macrophages, J. Leukoc. Biol. 70 (2) (2001) 163-170.
[38] D. Movita, K. Kreefft, P. Biesta, A. van Oudenaren, P. J. Leenen, H. L. Janssen, A. Boonstra, Kupffer cells express a unique combination of phenotypic and functional characteristics compared with splenic and peritoneal macrophages, J. Leukoc. Biol. 92 (4) (2012) 723-733.
[39] G. L. Su, R. D. Klein, A. Aminlari, H. Y. Zhang, L. Steinstraesser, W. H. Alarcon, D. G. Remick, S. C. Wang, Kupffer cell activation by lipopolysaccharide in rats: role for lipopolysaccharide binding protein and toll—like receptor 4, Hepatology 31 (4) (2000) 932-936.
[40] J. A. Bonzo, K. Rose, K. Freeman, E. Deibert, K. B. Amaral, S. S. Ferguson, M. E. Andersen, R. P. Witek, E. L. LeCluyse, Differential Effects of Trovafloxacin on TNF-α and IL-6 Profiles in a Rat Hepatocyte—Kupffer Cell Coculture System, Appl. In Vitro Toxicol. 1 (1) (2015) 45-54.
[41] C. Lin, S. Heyward, C. A. Brown, S. R. Khetani, Engineering Highly Functional Co-cultures of Cryopreserved Primary Human Hepatocytes and Kupffer Macrophages via Microfabrication, Drug Metab. Rev. (2015) 168-169.
[42] A. Geier, C. G. Dietrich, S. Voigt, S. K. Kim, T. Gerloff, G. A. Kullak-Ublick, J. Lorenzen, S. Matern, C. Gartung, Effects of proinflammatory cytokines on rat organic anion transporters during toxic liver injury and cholestasis, Hepatology 38 (2) (2003) 345-354.
[43] K. Sato, C. Hall, S. Glaser, H. Francis, F. Meng, G. Alpini, Pathogenesis of Kupffer cells in cholestatic liver injury, Am. J. Pathol. 186 (9) (2016) 2238-2247.
[44] S. Anthérieu, P. B. E. Azzi, J. Dumont, Z. Abdel-Razzak, C. Guguen-Guillouzo, B. Fromenty, M. A. Robin, A. Guillouzo, Oxidative stress plays a major role in chlorpromazine—induced cholestasis in human HepaRG cells, Hepatology 57 (4) (2013) 1518-1529.
[45] G. Zollner, P. Fickert, D. Silbert, A. Fuchsbichler, H.-U. Marschall, K. Zatloukal, H. Denk, M. Trauner, Adaptive changes in hepatobiliary transporter expression in primary biliary cirrhosis, J. Hepatol. 38 (6) (2003) 717-727.
[46] P. Knoll, J. Schlaak, A. Uhrig, P. Kempf, K.-H. M. zum Büschenfelde, G. Gerken, Human Kupffer cells secrete IL-10 in response to lipopolysaccharide (LPS) challenge, J. Hepatol. 22 (2) (1995) 226-229.
[47] R. P. Witek, N. Durham, Co-culture of Cryopreserved Primary Rat and Human Kupffer Cells and Hepatocytes to Study APAP Cytotoxicity, Drug Metab. Rev. (2011) 156-157.
[48] P. Walbrun, C. Hellerbrand, T. S. Weiss, S. Netter, D. Neumaier, E. Gaebele, R. Wiest, J. Schoelmerich, M. Froh, Characterization of rat and human Kupffer cells after cryopreservation, Cryobiology 54 (2) (2007) 164-172.
[49] J. A. Sunman, R. L. Hawke, E. L. LeCluyse, A. D. Kashuba, Kupffer cell-mediated IL-2 suppression of CYP3A activity in human hepatocytes, Drug Metab. Dispos. 32 (3) (2004) 359-363.
[50] T. V. Nguyen, O. Ukairo, S. R. Khetani, M. McVay, C. Kanchagar, W. Seghezzi, G. Ayanoglu, O. Irrechukwu, R. Evers, Establishment of a hepatocyte-Kupffer cell co-culture model for assessment of proinflammatory cytokine effects on metabolizing enzymes and drug transporters, Drug Metab. Dispos. 43 (5) (2015) 774-785.
[51] V. Kegel, E. Pfeiffer, B. Burkhardt, J. L. Liu, K. Zeilinger, A. K. Missler, D. Seehofer, G. Damm, Subtoxic concentrations of hepatotoxic drugs lead to Kupffer cell activation in a human in vitro liver model: An approach to study DILI, Mediators Inflamm. (2015) 640631.
[52] M. Melino, V. L. Gadd, G. V. Walker, R. Skoien, H. D. Barrie, D. Jothimani, L. Horsfall, A. Jones, M. J. Sweet, G. P. Thomas, Macrophage secretory products induce an inflammatory phenotype in hepatocytes, World J. Gastroenterol. 18 (15) (2012) 1732-1744.
[53] M. McVay, C. Kanchagar, S. R. Khetani, 0. Ukairo, In vitro modeling of cytokine—drug interactions using micropatterned co-cultures of primary hepatocytes and Kupffer macrophages, Toxicol. Lett. (221) (2013) S150.

In this document and in its claims, the verb “to comprise” and its conjugations is used in its non-limiting sense to mean that items following the word are included, but items not specifically mentioned are not excluded. In addition, reference to an element by the indefinite article “a” or “an” does not exclude the possibility that more than one of the element is present, unless the context clearly requires that there be one and only one of the elements. The indefinite article “a” or “an” thus usually means “at least one”.

Each of the applications and patents mentioned in this document, and each document cited or referenced in each of the above applications and patents, including during the prosecution of each of the applications and patents (“application cited documents”) and any manufacturer's instructions or catalogues for any products cited or mentioned in each of the applications and patents and in any of the application cited documents, are hereby incorporated herein by reference. Furthermore, all documents cited in this text, and all documents cited or referenced in documents cited in this text, and any manufacturer's instructions or catalogues for any products cited or mentioned in this text, are hereby incorporated herein by reference.

Various modifications and variations of the described methods and system of the invention will be apparent to those skilled in the art without departing from the scope and spirit of the invention. Although the invention has been described in connection with specific preferred embodiments, it should be understood that the invention as claimed should not be unduly limited to such specific embodiments. Indeed, various modifications of the described modes for carrying out the invention which are obvious to those skilled in molecular biology or related fields are intended to be within the scope of the claims.

1. A method of producing an iPSC-derived Kupffer Cell (iKC) comprising the steps of:
(a) providing a macrophage precursor (preMcp) derived from an induced pluripotent stem cell (iPSC);
(b) culturing the macrophage precursor (preMcp) in the presence of a hepatic cue; and
(c) obtaining an iPSC-derived Kupffer Cell (iKC) therefrom;
wherein the iPSC-derived Kupffer Cell (iKC) displays a biological property of a primary adult human KC (pKC).

2. The method according to claim 1, in which the hepatic cue comprises contacting the macrophage precursor with primary human hepatocyte conditioned media (PHCM) and Advanced DMEM.

3. The method according to claim 1, in which the biological property is selected from the group consisting of:
(a) expression of a macrophage marker;
(b) phagocytosis;
(c) release of an inflammatory cytokine, growth factor or reactive oxygen species upon activation; or
(d) secretion of IL-6 and TNFα upon stimulation with LPS.

4. The method according to claim 3, in which the biological activity comprises expression of a macrophage marker and:
(a) the macrophage marker is selected from the group consisting of: CD11 (GenBank Accession Number NM_000632.3), CD14 (GenBank Accession Number NM_001174105.1), CD68 (GenBank Accession Number NM_001251.2), CD 163 (GenBank Accession Number NM_203416.3) and CD32 (GenBank Accession Number NM_001136219.1); or
(b) the macrophage marker is selected from CLEC-4F (GenBank Accession Number NM_173535.2), ID1 (GenBank Accession Number NM_181353.2) and ID3 (GenBank Accession Number NM_002167.4).

5. The method according to claim 1, in which the macrophage precursor (preMcp) is derived from an induced pluripotent stem cell (iPSC) by:
(a) culturing the induced pluripotent stem cell (iPSC) to generate an embryoid body (EB); and
(b) culturing the embryoid body (EB) to generate a macrophage precursor (preMcp) cell.

6. The method according to claim 5, in which step (a) comprises contacting the iPSC with bone morphogenetic protein-4 (BMP-4, GenBank Accession Number Q53XC5), vascular endothelial growth factor (VEGF, GenBank Accession Number NP_001165097), stem cell factor (SCF, GenBank Accession Number P21583.1) and ROCK Inhibitor.

7. The method according to claim 5, in which step (b) comprises contacting the embryoid body with macrophage colony stimulating factor (M-CSF, GenBank Accession Number P09603) Interleukin-3 (IL-3, GenBank Accession Number AAC08706), glutamax and β-mercaptoethanol.

8. The method according to claim 1, in which the induced pluripotent stem cell (iPSC) is a MYB-independent iPSC.

9. (canceled)

10. A composition comprising a human iKC prepared by the method of claim 1 and an hepatocyte.

11. A method for determining the hepatotoxicity of a drug, the method comprising contacting an iKC or a composition of claim 10 with the drug.

12.-15. (canceled)

16. A method of treatment or prevention of a liver disease or condition, the method comprising administering or transplanting an iPSC-derived Kupffer Cell (iKC) produced by the method of claim 1 to a patient in need of such treatment.

17. The method of claim 6, wherein the iPSC is contacted with BMP-4 at 50 ng/mL, VEGF at 50 ng/mL, SCF at 20 ng/mL and ROCK Inhibitor at 10 mM.

18. The method of claim 7, wherein the embryoid body is contacted with M-CSF at 100 ng/mL, IL-3 at 25 ng/mL, glutamax at 2 mM and β-mercaptoethanol at 0.055 mM.

19. The composition of claim 10, wherein the hepatocyte is a primary human hepatocyte (pHeP) or an iPSC-derived hepatocyte (iHep).

20. The composition of claim 19, wherein the iKC and the iHep are derived from the same stem cell source.

21. The method of claim 11, wherein the drug is selected from the group consisting of: an inflammation-associated drug, Acetaminophen, Trovafloxacin, and Chlorpromazine.

22. A model for a liver disease or condition, the model comprising a composition of claim 10.

23. The model of claim 22, wherein the disease or condition is selected from the group consisting of: liver injury, drug-induced liver injury (DILI), liver disease, steatohepatitis, cholestasis, liver fibrosis and viral hepatitis.
